(19) United States
(12) Patent Application Publication
(10) Pub. No.: US 2010/0093648 A1
(43) Pub. Date: Apr. 15, 2010

(54) TREATMENT OF MACULAR DEGENERATION-RELATED DISORDERS

(76) Inventor: Antonio CRUZ, Toronto (CA)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/576,957

(22) Filed: Oct. 9, 2009

Related U.S. Application Data
(60) Provisional application No. 61/104,094, filed on Oct. 9, 2008.

(51) Int. Cl. A61K 31/7004; A61P 27/02

(52) U.S. Cl. 514/23

(57) ABSTRACT

The invention relates to compositions and methods for preventing or treating a macular degeneration-related disorder, comprising a scyllo-inositol compound or pharmaceutically acceptable salts thereof.

This application claims the benefit of U.S. Provisional Application No. 61/104,094, filed Oct. 9, 2008, the disclosure of which is incorporated herein by reference in its entirety.

### FIELD OF THE INVENTION

The invention relates to compositions, methods and treatments for macular degeneration-related disorder.

### BACKGROUND OF THE INVENTION

Macular degeneration is a leading cause of blindness affecting the aged population in the industrialized world. The disease has a significant impact on the physical and mental health of the aged population and is becoming a major public health burden. The disease is characterized by a progressive loss of central vision resulting from degenerative and neovascular changes in the macula, the specialized region in the retina responsible for fine visual acuity. There are two forms of the disease, the more common nonexudative (dry) form, and the less prevalent, late-stage exudative or “wet” form. Palliative treatment options for the wet form of the disease include anti-neovascular agents (e.g. Macugen®, pegaptanib sodium, and Lucentis®, ranibizubmab), photodynamic therapy and laser surgery. There are no current therapies for the dry form of macular degeneration but antioxidants have been reported to delay or possibly prevent the transition from intermediate to advanced disease.

### SUMMARY OF THE INVENTION

The present invention generally relates to methods for treating a macular degeneration-related disorder in a subject comprising administering a therapeutically effective amount of a scyllo-inositol compound. The methods of the invention can be used therapeutically or prophylactically in a subject susceptible to or suffering from a macular degeneration-related disorder.

In embodiments of the invention, methods are provided for treating a macular degeneration-related disorder in a subject comprising administering to the subject an effective amount of a scyllo-inositol compound, or a pharmaceutically acceptable salt thereof, or a medicament comprising a scyllo-inositol compound and a pharmaceutically acceptable carrier, excipient, or vehicle, which results in therapeutic or prophylactic effects following treatment. In some embodiments, the invention relates to a method for the treatment of a subject suffering from a macular degeneration-related disorder comprising administering a scyllo-inositol compound or a pharmaceutically acceptable salt thereof, to the subject in a therapeutically effective amount to treat the subject. In some embodiments, the invention relates to methods for treating or preventing the development of a macular degeneration-related disorder, in a subject suffering from or at risk of developing a macular degeneration-related disorder comprising administering to the subject an effective amount of a scyllo-inositol compound.

In an embodiment, the method is provided for treating a macular degeneration-related disorder comprising administering an effective amount of a scyllo-inositol compound and not administering phytic acid, or a phytate salt, or hydrolysate of phytic acid or hexakisphosphate myo-inositol, hexakisphosphate scyllo-inositol, hexakisphosphate D-chiro-inositol, hexakisphosphate L-chiro-inositol, hexakisphosphate neo-inositol and hexakisphosphate muco-inositol.

In an embodiment, the invention relates to a method of treatment comprising administering a therapeutically effective amount of a scyllo-inositol compound, a pharmaceutically acceptable salt thereof, or a medicament comprising a scyllo-inositol compound, and a pharmaceutically acceptable carrier, excipient, or vehicle, which upon administration to a subject with symptoms of a macular degeneration-related disorder produces therapeutic effects. In particular embodiments of the invention, therapeutic effects may be evidenced by an improvement in visual acuity. In an embodiment of the invention, therapeutic effects are evidenced by a subject losing less than 15 letters of visual acuity from baseline after administration.

In an embodiment, the invention provides a method for amelioriating symptoms or onset of a macular degeneration-related disorder comprising administering an effective amount for amelioriating symptoms or onset of a macular degeneration-related disorder of a scyllo-inositol compound, a pharmaceutically acceptable salt thereof, or a medicament comprising a scyllo-inositol compound and a pharmaceutically acceptable carrier, excipient, or vehicle.

In an embodiment, the invention provides a method for amelioriating progression of a macular degeneration-related disorder comprising administering an effective amount (e.g., a therapeutically effective amount) for amelioriating progression of the disorder, of a scyllo-inositol compound, a pharmaceutically acceptable salt thereof, or a medicament comprising a scyllo-inositol compound and a pharmaceutically acceptable carrier, excipient, or vehicle.

In an embodiment, the invention provides a method for amelioriating progression of age-related macular degeneration (AMD), or progression of dry AMD to wet AMD, comprising administering an effective amount (e.g. a therapeutically effective amount) of a scyllo-inositol compound, a pharmaceutically acceptable salt thereof, or a medicament or pharmaceutical composition comprising a scyllo-inositol compound and a pharmaceutically acceptable carrier, excipient, or vehicle.

In an embodiment, the invention relates to a method of delaying the onset or progression of a macular degeneration-related disorder comprising administering an effective amount (e.g a therapeutically effective amount) for delaying the onset or progression of the disorder of a scyllo-inositol compound, a pharmaceutically acceptable salt thereof, or a medicament comprising a scyllo-inositol compound and a pharmaceutically acceptable carrier, excipient, or vehicle.

In an embodiment, the invention relates to a method of delaying the onset or progression of AMD or onset or progression of dry AMD to wet AMD, comprising administering an effective amount of a scyllo-inositol compound, a pharmaceutically acceptable salt thereof, or a medicament comprising a scyllo-inositol compound and a pharmaceutically acceptable carrier, excipient, or vehicle.

In an embodiment, the invention relates to a method of preventing a macular degeneration-related disorder in a subject comprising administering a prophylactically effective amount of a scyllo-inositol compound, a pharmaceutically acceptable salt thereof, or a medicament comprising a prophylactically effective amount of a scyllo-inositol compound and a pharmaceutically acceptable carrier, excipient, or vehicle.

The invention provides a method of treating a subject at risk of developing a macular degeneration-related disorder comprising the steps of: a) identifying a subject at risk of developing a macular degeneration-related disorder; and b) administering to the subject an amount of a scyllo-inositol compound effective to inhibit or delay onset of the disorder.

In an embodiment, the invention provides a method for protecting ocular tissues (e.g. macular cells) in a subject having a macular degeneration-related disorder comprising administering a prophylactically effective amount of a scyllo-inositol compound, a pharmaceutically acceptable salt thereof, or a medicament comprising a prophylactically effective amount of a scyllo-inositol compound and a pharmaceutically acceptable carrier, excipient, or vehicle.

In an embodiment, the invention provides a method for administering a scyllo-inositol compound or a medicament comprising a scyllo-inositol compound and a pharmaceutically acceptable carrier, excipient, or vehicle in an effective amount (e.g., therapeutically effective amount) to patients who need treatments for a macular degeneration-related disorder while minimizing the occurrence of adverse effects or side-effects.

In an embodiment, the invention provides medicaments or pharmaceutical compositions for prevention and/or treatment of macular degeneration-related disorders. Thus, the invention further contemplates a medicament comprising a scyllo-inositol compound, in particular a therapeutically effective amount of a scyllo-inositol compound, for treating macular degeneration-related disorders. More particularly, the invention in embodiments contemplates a medicament in a form adapted for administration to a subject to provide therapeutic effects to treat macular degeneration-related disorders. In an embodiment, a medicament is in a form such that administration to a subject suffering from a macular degeneration-related disorder results in an improvement in visual acuity and/or, slowing or arrest of the progress of a macular degeneration-related disorder.

In embodiments, the invention also features a medicament or pharmaceutical composition comprising a scyllo-inositol compound, in particular a therapeutically effective amount of a scyllo-inositol compound, for improving visual acuity. A medicament or pharmaceutical composition of the invention can be in a pharmaceutically acceptable carrier, excipient, or vehicle. In an embodiment, a medicament or pharmaceutical composition is provided comprising a scyllo-inositol compound in a suitable pH, osmolality, tonicity, purity and sterility to allow safe administration to a subject.

A scyllo-inositol compound or medicament comprising a scyllo-inositol compound can be administered to a patient by any route effective to treat a macular degeneration-related disorder, in particular an ocular of systemic route.

In embodiments, the invention additionally provides a method of preparing a stable medicament comprising one or more scyllo-inositol compound in an effective amount for treating a macular degeneration-related disorder. After medicaments have been prepared, they can be placed in an appropriate container and labeled for treatment of a macular degeneration-related disorder. For administration of a medicament of the invention, such labeling would include amount, frequency, and method of administration.

In embodiments, the invention also contemplates the use of at least one scyllo-inositol compound for treating a macular degeneration-related disorder, or for the preparation of a medicament for treating a macular degeneration-related disorder. In embodiments, the invention additionally provides uses of a scyllo-inositol compound for the prevention of a macular degeneration-related disorder or in the preparation of medicaments for the prevention of a macular degeneration-related disorder.

In embodiments, the invention provides a method of treating a macular degeneration-related disorder in a subject non-responsive to a conventional treatment, in particular non-responsive to an anti-vascular endothelial growth factor (anti-VEGF) therapeutic, an anti-oxidant, photocoagulation therapy and/or photodynamic therapy, comprising administering to the patient a therapeutically effective amount of a scyllo-inositol compound.

In an embodiment, the invention provides a method of treating a macular degeneration-related disorder in a subject that has had a sub-optimal response to treatment with an anti-vascular endothelial growth factor (anti-VEGF) therapeutic, an anti-oxidant, photocoagulation therapy and/or photodynamic therapy, comprising administering to the patient an effective amount of a scyllo-inositol compound and optionally, an anti-vascular endothelial growth factor (anti-VEGF) therapeutic, an anti-oxidant, photocoagulation therapy or photodynamic therapy, to provide an enhanced or optimal response.

In embodiments, the invention contemplates a method of retreatment using a therapeutically effective amount of a scyllo-inositol compound for subjects suffering from a macular degeneration-related disorder who fail to respond to therapy with an anti-vascular endothelial growth factor (anti-VEGF) therapeutic, an anti-oxidant, photocoagulation therapy or photodynamic therapy, or who following cessation of such therapy suffer a relapse or who relapse while on therapy. In an embodiment of the invention, the subjects to be treated have experienced at least one relapse during the year preceding the beginning of the treatment. In other embodiments of the invention, refractory subjects to be treated have experienced at least one relapse and developed increased disability because of the disorder.

In embodiments, the invention also contemplates a method of retreatment using a therapeutically effective amount of a scyllo-inositol compound for a subject suffering from a macular degeneration-related disorder that has one or more adverse events with an anti-vascular endothelial growth factor (anti-VEGF) therapeutic, an anti-oxidant, photocoagulation therapy or photodynamic therapy. Adverse envents include without limitation endophthalmitis, retinal detachment, iatrogenic traumatic cataract, conjunctival hemorrhage, eye pain, and/or vitreous disorders.

In embodiments, the invention includes combination treatments comprising administering a first agent comprising a scyllo-inositol compound, and a second agent comprising an anti-vascular endothelial growth factor (anti-VEGF) therapeutic, an anti-oxidant, photocoagulation therapy and/or photodynamic therapy. In embodiments, the responses to, or effects of, the first agent and second agent are additive (i.e., the responses or effects are equal to the sum of the responses or effects of the two individual agents). In other embodiments, the responses to, or effects of, the first agent and second agent are greater than either agent given individually.

In embodiments, the invention includes combination treatments providing synergistic activity or delivering synergistically effective amounts of a scyllo-inositol compound, and a second agent such as an anti-vascular endothelial growth factor (anti-VEGF) therapeutic, an anti-oxidant, photocoagulation therapy and/or photodynamic therapy. Combination treatments which may be suitable for use in the present invention include those wherein the treatments are administered in synergistically effective dosages or in the case of active ingredients are in compositions comprising synergistically effective amounts of the active ingredients. Such a combination treatment or composition comprises sufficient amounts of each component to achieve a desired result. In embodiments the desired result is greater than the result achieved with each component on its own.

In embodiments, the invention relates to synergistically effective amounts of a scyllo-inositol compound and an anti-vascular endothelial growth factor (anti-VEGF) therapeutic or an anti-oxidant for use in treating a macular degeneration-related disorder or in the preparation of a medicament for treating such a disorder.

In embodiments, the invention also provides a method of treating a subject suffering from a macular degeneration-related disorder and receiving a conventional treatment comprising administering to the patient a therapeutically effective amount of a scyllo-inositol compound, and wherein the dosage of the conventional treatment is decreased or reduced. In embodiments of the invention the conventional treatment includes an anti-vascular endothelial growth factor (anti-VEGF) therapeutic, an anti-oxidant, photocoagulation therapy and/or photodynamic therapy.

In embodiments, the invention also provides a kit comprising a scyllo-inositol compound, or a medicament comprising same. In an embodiment, the invention provides a kit for preventing and/or treating a macular degeneration-related disorder containing a medicament comprising a scyllo-inositol compound, a container, and instructions for use. A composition of the kit can further comprise a pharmaceutically acceptable carrier, excipient, or vehicle and/or a second agent. In an embodiment, the invention provides a method of promoting sales of a medicament or kit of the invention comprising the public distribution of information that administration of the medicament or kit is associated with treatment or prophylaxis of a macular degeneration-related disorder.

These and other aspects, features, and advantages of the present invention should be apparent to those skilled in the art from the following drawings and detailed description.

### DESCRIPTION OF THE DRAWINGS

FIG. 1 is a graph showing scotopic b-wave amplitudes of a normal diet group (ND blue group), a group receiving a high cholesterol diet plus scyllo-inositol (AZD-103/ELN005) in their drinking water (HFC red/green group) and a group receiving a high cholesterol diet plus regular drinking water (HFC yellow group).

### DETAILED DESCRIPTION OF EMBODIMENTS

All technical and scientific terms used herein have the same meaning as commonly understood by one of ordinary skill in the art to which this invention belongs. For convenience, certain terms employed in the specification, examples, and appended claims are collected here. The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”. The term “about” means plus or minus 0.1 to 50%, 5-50%, or 10-40%, preferably 10-20%, more preferably 10% or 15%, of the number to which reference is being made. Further, it is to be understood that “a”, “an”, and “the” include plural referents unless the content clearly dictates otherwise. Thus, for example, reference to a compound includes a mixture of two or more compounds.

The terms “administering” and “administration” refer to the process by which an effective amount of a scyllo-inositol compound or medicament contemplated herein is delivered, for any period of time, to a subject for prevention and/or treatment purposes. The scyllo-inositol compounds and medicaments are administered in accordance with good medical practices taking into account the subject's clinical condition, the site and method of administration, dosage, patient age, sex, body weight, and other factors known to physicians.

The term “treating” refers to reversing, alleviating, inhibiting or delaying the progress of a disease, one or more symptoms of such disease, or disabilities associated with such disease. In embodiments of the invention, treating includes the management and care of a subject at diagnosis or later. A treatment may be either performed in an acute or chronic way. Depending on the condition of the subject, the term in some embodiments may refer to “preventing” a disease, and includes preventing the onset of a disease, or preventing the symptoms associated with a disease. The term in some embodiments also refers to reducing the severity of a disease or symptoms associated with such disease prior to affliction with the disease. Such prevention or reduction of the severity of a disease prior to affliction refers to administration of a scyllo-inositol compound, or medicament comprising same, to a subject that is not at the time of administration afflicted with the disease. “Preventing” in some embodiments also refers to preventing the recurrence of a disease or of one or more symptoms associated with such disease. In some embodiments, a treatment is used to combat a disease and includes administration of the active compounds to prevent or delay the onset of the symptoms or complications, or alleviating the symptoms or complications, or eliminating or partially eliminating the disease. The terms “treatment”, “therapeutic”, “therapeutically” refer to the act of treating, as “treating” is defined above. The terms “prevention”, “prophylatic”, “prophylatically” refer to the act of preventing, as “preventing” is defined above.

The terms “subject”, “individual”, and “patient” are used interchangeably herein and refer to an animal including a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. Among other specific embodiments a mammal of the present invention can be Canis familiaris (dog), Felis catus (cat), Elephas maximus (elephant), Equus caballus (horse), Sus domesticus (pig), Camelus dromedarious (camel), Cervus axis (deer), Giraffa camelopardalis (giraffe), Bos taurus (cattle/cows), Capra hircus (goat), Ovis aries (sheep), Mus musculus (mouse), Lepus brachyurus (rabbit), Mesocricetus auratus (hamster), Cavia porcellus (guinea pig), Meriones unguiculatus (gerbil), or Homo sapiens (human). In particular embodiments, the mammal is a human. Typical subjects for treatment include persons afflicted with or suspected of having or being pre-disposed to a macular degeneration-related disorder, or persons susceptible to, suffering from or that have suffered from a macular degeneration-related disorder. A subject may or may not have a genetic predisposition for a macular degeneration-related disorder. In particular aspects, a subject shows symptoms of a macular degeneration-related disorder. In embodiments of the invention, the subjects are susceptible to, or suffer from a macular degeneration-related disorder.

In an aspect of the invention, the subject is a human subject with macular degeneration-related disorder in one or both eyes.

In aspects of the invention the subject is a human subject receiving conventional therapy, in particular anti-VEGF therapy, more particularly Macugen or Lucentis

In aspects of the invention, a subject has choroidal neovascularization secondary to age-related macular degeneration.

In aspects of the invention, a subject has dry age-related macular degeneration.

In aspects of the invention, a subject is a patient diagnosed with age-related macular degeneration. Methods of diagnosis of age-related macular degeneration disorders are disclosed herein. Diagnostic methods known in the art can also be employed including (a) measurement of visual acuity, for example, using a Snellen chart, a Bailey-Louie chart, a decimal progression chart, a Freiburg visual acuity test, measurement of angle of resolution, and the like; (b) measurement of metamorphopsia (visual distortion) using an Amsler chart; (c) measurement of contrast sensitivity using a Pelli-Robson chart; and (d) standard opthalmologic examination of the fundus, stereo biomicroscopic examination of the macula, intravenous fundus fluorescein angiography, fundus photography, indocyanine green video-angiography and optical coherence tomography.

In embodiments, the diagnosis of age-related macular degeneration may be based upon the presence of visual disturbance and characteristic findings on dilated eye examination. The dry type of age-related macular degeneration may be characterized by the presence of drusens on dilated eye examination, round or oval patches of geographic atrophy of the retina evident as areas of depigmentation, and increased pigmentation with RPE pigmentary mottling. The wet type of age-related macular degeneration may be characterized by subretinal fluid, hemorrhage and/or lipid exudates on dilated eye examination, and neovascularization appearing as a grayish discoloration in the macular area. (See, for example, Age-Related Macular Degeneration, Jennifer Lim, Ed., 2^{nd} Edition, Informa Healthcare USA, 2008 and Age-Related Macular Degeneration, Holz, Pauliekoff, Spaide and Bird, Editors, Springer-Verlag, Heidelberg, 2004, for information on age-related macular degeneration.)

In aspects of the invention, a subject is a newly diagnosed or uniocular patient.

In particular aspects, a subject has one or more of the following pathologies: soft distinct drusen (≧63 μM); soft indistinct drusen (≧125 μM) or reticular drusen only, soft indistinct drusen (≧125 μM) or reticular drusen with pigmentation abnormalities; and atrophic or neovascular AMD. [See van Leeuwen et al, Arch Opthalmol. 2003, 121 (4):519-26.]

In particular aspects, a subject has one or more of the following pathologies: hard drusen (<63 μM) only, pigmentation abnormalities only, no soft drusen (>63 μM) and soft distinct drusen (≧63 μM) with pigmentation abnormalities.

In aspects of the invention, a subject has neovascular age-related macular degeneration characteristics including classic, occult and mixed lesions of up to 12 disc areas and baseline visual acuity in an eye between 20/40 and 20/320.

In aspects of the invention, a subject has one or more of the following: visual acuity loss of greater than 5 letters, optical coherence tomography (OCT) evidence of fluid in the macula, an increase in OCT central retinal thickness of greater than 100 mM, existing or new macular hemorrhage, existing or new area of classic choroidal neovascularization, and persistent fluid (by OCT), in particular persistent fluid greater than 1 month after treatment (with a conventional therapy).

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular medicament. Examples of carriers etc. include but are not limited to saline, buffered saline, dextrose, water, glycerol, ethanol, and combinations thereof. The use of such media and agents for an active substance is well known in the art. Acceptable carriers, excipients or vehicles may be selected from any of those commercially used in the art.

“Pharmaceutically acceptable salt(s),” means a salt that is pharmaceutically acceptable and has the desired pharmacological properties. By pharmaceutically acceptable salts is meant those salts which are suitable for use in contact with the tissues of a subject or patient without undue toxicity, irritation, allergic response and the like, and are commensurate with a reasonable benefit/risk ratio. Pharmaceutically acceptable salts are described for example, in S. M. Berge, et al., J. Pharmaceutical Sciences, 1977, 66:1. Suitable salts include salts that may be formed where acidic protons in compounds are capable of reacting with inorganic or organic bases. Suitable inorganic salts include those formed with alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Suitable salts also include acid addition salts formed with inorganic acids (e.g. hydrochloric and hydrobromic acids) and organic acids (e.g. acetic acid, citric acid, maleic acid, and the alkane- and arene-sulfonic acids such as methanesulfonic acid and benezenesulfonic acid). When there are two acidic groups present, a pharmaceutically acceptable salt may be a mono-acid-mono-salt or a di-salt; and similarly where there are more than two acidic groups present, some or all of such groups can be salified.

“Effective amount” relates to the amount or dose of a scyllo-inositol compound or medicament thereof that will lead to one or more desired effects, in particular, one or more therapeutic or prophylatic effects, or an amount that is effective in treating a macular degeneration related-disorder. An effective amount of a substance can vary according to factors such as the disease state, age, sex, and weight of the subject, and the ability of the substance to elicit a desired response in the subject. A dosage regimen may be adjusted to provide the optimum response (e.g. therapeutic or prophylactic effects). For example, several divided doses may be administered daily or the dose may be proportionally reduced as indicated by the exigencies of the therapeutic situation. In aspects of the invention, an effective amount reduces or prevents vision loss.

In embodiments, the effective amount is a “therapeutically effective amount”. A “therapeutically effective amount” includes an amount of an active ingredient sufficient to reverse, alleviate, inhibit or delay the progress of a disorder, one or more symptoms of such disorder, or disabilities associated with such disorder. In certain embodiments, the term refers to an amount of an active ingredient that improves one or more of the symptoms of the disorder being treated as compared to those symptoms that occur without treatment. An improvement may be permanent or temporary.

In embodiments, the effective amount is a “prophylatically effective amount”. The term “prophylactically effective amount” includes an amount effective, at dosages and for periods of time necessary, to achieve a desired prophylactic result, i.e. an amount sufficient to result in the prevention of onset or recurrence of a disorder or one or more symptoms of a disorder. Since a prophylactic dose is used in subjects prior to or at an earlier stage of disease, the prophylactically effective amount may be less than the therapeutically effective amount.

The term “pure” in general means better than 90%, 92%, 93%, 94%, 95%, 96%, 97%, 98% or 99% pure, and “substantially pure” means a compound synthesized such that the compound, as made available for consideration into a method or medicament of the invention, has only those impurities that can not readily nor reasonably be removed by conventional purification processes.

“Optional” or “optionally” means that the subsequently described event or circumstance may but need not occur, and that the description includes instances where the event or circumstance occurs and instances in which it does not occur. For example, “optionally substituted” means that a radical may but need not be substituted, and the description includes situations where the radical is substituted and situations where the radical is not substituted.

“Synergistic” means a greater pharmacological or therapeutic effect with the use of a multi-component composition or combination therapy of a scyllo-inositol compound and a conventional therapy or second agent such as an anti-vascular endothelial growth factor (anti-VEGF) therapeutic, an anti-oxidant, photocoagulation therapy or photodynamic therapy, than with the use of any of these treatments individually or alone. This synergistic effect can work through either similar or different mechanisms or pathways of action. One potential advantage of a combination therapy with a synergistic effect is that standard dosages may be used for a greater therapeutic effect than expected from the addition of the effect of one or two treatments administered alone; or alternatively lower dosages or reduced frequency of administration of the treatments may be used to achieve a better therapeutic effect.

“Antibody” includes any immunoglobulin or a derivative thereof which maintains binding ability, or any protein having a binding domain which is homologous or largely homologous to an immunoglobulin binding domain. An antibody may be derived from natural sources, or partly or wholly synthetically produced (e.g., using recombinant DNA techniques, chemical synthesis, etc.). An antibody can be of any species, e.g., human, rodent, rabbit, goat, chicken, etc. and it may be a member of any immunoglobulin class, including any of the human classes: IgG, IgM, IgA, IgD, and IgE. An antibody includes a fragment of an antibody such as an Fab′, F(ab′)₂, scFv (single-chain variable) or other fragment that retains an antigen binding site, a recombinantly produced scFv fragment, including recombinantly produced fragments, and a monovalent, bivalent or multivalent antibody. An antibody may be a chimeric or partially or completely “humanized” antibody in which, for example, a variable domain of mouse origin is fused to a constant domain of human origin, thus retaining the specificity of the mouse antibody. The domain of human origin may be first synthesized in a human or it may be generated in rodents whose genome incorporates human immunoglobulin genes. (See, e.g., Vaughan, et al., (1998), Nature Biotechnology, 16: 535-539.) An antibody may be polyclonal or monoclonal, Methods for producing antibodies that specifically bind to molecules of interest are known in the art.

“Ocular tissue” refers to a tissue contained within the eye. Ocular tissue includes without limitation tissues comprising cells of the lens, the cornea (endothelial, stromal and/or epithelial corneal cells), the iris, the retina, choroid, sclera, ciliary body, vitrous body, ocular vasculature, canal of Schlemm, ocular muscle cells, optic nerve, and other ocular sensory, motor and autonomic nerves. In aspects of the invention, the term refers to tissues comprising cells of the macula or macular cells.

A “scyllo-inositol compound” includes a compound having the structure of the formula Ia or Ib:

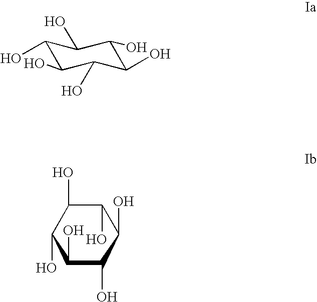

A scyllo-inositol compound also includes a compound of the formula Ia or Ib wherein one or more, preferably two or three, more preferably one or two, hydroxyl groups are replaced by substituents, in particular univalent substituents, with retention of configuration. In aspects of the invention, a scyllo-inositol compound comprises a compound of the formula Ia or Ib wherein one or two, most preferably one, hydroxyl group is replaced by univalent substituents, with retention of configuration. Suitable substituents include without limitation hydrogen; alkyl; substituted alkyl; acyl; alkenyl; substituted alkenyl; alkynyl; substituted alkynyl; alkoxy; substituted alkoxy; halogen; —NHR¹ wherein R¹ is hydrogen, acyl, alkyl or —R²R³ wherein R² and R³ are the same or different and represent acyl or alkyl; —PO₃H₂; —SR⁴ wherein R⁴ is hydrogen, alkyl, or —O₃H; and —OR⁵ wherein R⁵ is —SO₃H.

The terms used herein for radicals including “alkyl”, “alkoxy”, “alkenyl”, “alkynyl”, “hydroxyl” etc, refer to optionally substituted radicals, i.e, both unsubstituted and substituted radicals. The term “substituted,” as used herein, means that any one or more moiety on a designated atom (e.g., hydroxyl) is replaced with a selected group provided that the designated atom's normal valency is not exceeded, and that the substitution results in a stable compound. Combinations of substituents and/or radicals are permissible only if such combinations result in stable compounds. “Stable compound” refers to a compound that is sufficiently robust to survive isolation to a useful degree of purity from a reaction mixture, and formulation into an efficacious therapeutic agent.

“Alkyl” means a monovalent, saturated hydrocarbon radical which may be a straight chain (i.e. linear) or a branched chain. In certain aspects of the invention, an alkyl radical comprises from about 1 to 10, about 1 to 8, about 3 to 8, about 1 to 6, or about 1 to 3 carbon atoms. In certain embodiments of the invention an alkyl radical is a C₁-C₆ lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, n-hexyl, and the like, along with branched variations thereof. In some embodiments, a “substituted alkyl” includes an alkyl group substituted by, for example, one to five substituents, and in certain embodiments 1 to 3 substituents, such as alkyl, alkoxy, oxo, alkanoyl, alkanoyloxy, acyl, amino, hydroxyamino, alkylamino, alkoxyamino, halogen, hydroxyl, carboxyl, carbamyl, carboxylalkyl, keto, thioketo, thiol, alkylthiol, sulfonamide, thioalkoxy, and nitro.

The term “alkenyl” refers to an unsaturated, acyclic branched or straight-chain hydrocarbon radical comprising at least one double bond. Alkenyl radicals may contain from about 2 to 10 carbon atoms and in some embodiments from about 3 to 8 carbon atoms, about 3 to 6 carbon atoms or about 2 to 6 carbon atoms. Examples of suitable alkenyl radicals include ethenyl, propenyl such as prop-1-en-1-yl, prop-1-en-2-yl, prop-2-en-1-yl (allyl), prop-2-en-2-yl, buten-1-yl, but-1-en-2-yl, 2-methyl-prop-1-en-1-yl, but-2-en-1-yl, but-2-en-2-yl, buta-1,3-buta-1,3-dien-2-yl, hexen-1-yl, 3-hydroxyhexen-1-yl, hepten-1-yl, and octen-1-yl, and the like. In some embodiments, the alkenyl groups include ethenyl (—CH═CH₂), n-propenyl (—CH₂CH═CH₂), iso-propenyl (—C(CH₃)═CH₂), and the like. An alkenyl radical may be optionally substituted similar to alkyl. In some embodiments, a “substituted alkenyl” includes an alkenyl group substituted by, for example, one to three substituents or one to two substituents, such as alkyl, alkoxy, haloalkoxy, alkylalkoxy, haloalkoxyalkyl, alkanoyl, alkanoyloxy, acyl, acylamino, acyloxy, amino, alkylamino, alkanoylamino, aminoacyl, aminoacyloxy, halogen, hydroxyl, carboxyl, carboxylalkyl, carbamyl, keto, thioketo, thiol, alkylthio, sulfonyl, sulfonamido, thioalkoxy, nitro, and the like.

The term “alkynyl” refers to an unsaturated, branched or straight-chain hydrocarbon radical comprising one or more triple bonds. Alkynyl radicals may contain about 2-10 carbon atoms or about 3 to 8 carbon atoms, and in some embodiments about 3 to 6 carbon atoms. In embodiments of the invention, “alkynyl” refers to straight or branched chain hydrocarbon groups of 2 to 6 carbon atoms having one to four triple bonds. Examples of suitable alkynyl radicals include ethynyl, propynyls, such as prop-1-yn-1-yl, prop-2-yn-1-yl, butynyls such as but-1-yn-1-yl, but-1-yn-3-yl, and but-3-yn-1-yl, pentynyls such as pentyn-1-yl, pentyn-2-yl, and 4-methoxypentyn-2-yl, and 3-methylbutyn-1-yl, and hexynyls such as hexyn-1-yl, hexyn-2-yl, and hexyn-3-yl and the like. A “substituted alkynyl” includes an alkynyl group substituted by, for example, a substituent, such as, alkyl, alkoxy, alkanoyl, alkanoyloxy, acyl, acylamino, acyloxy, amino, alkylamino, alkanoylamino, aminoacyl, aminoacyloxy, halogen, hydroxyl, carboxyl, carboxylalkyl, carbamyl, keto, thioketo, thiol, alkylthio, sulfonyl, sulfonamido, thioalkoxy, nitro, and the like.

As used herein, “halogen” or “halo” refers to fluoro, chloro, bromo and iodo. In embodiments, “halogen’ or “halo” refers to fluoro or chloro.

The term “hydroxyl” or “hydroxy” refers to a single —OH group.

The term “alkoxy” refers to a linear or branched oxy-containing radical having an alkyl portion of one to about ten carbon atoms, which may be substituted. Particular alkoxy radicals are “lower alkoxy” radicals having about 1 to 6, about 1 to 4 or about 1 to 3 carbon atoms. An alkoxy having about 1 to 6 carbon atoms includes a C₁-C₆ alkyl-O— radical wherein C₁-C₆ alkyl has the meaning set out herein. Illustrative examples of alkoxy radicals include without limitation methoxy, ethoxy, propoxy, butoxy, isopropoxy and tert-butoxy. An “alkoxy” radical may optionally be further substituted with one or more substitutents disclosed herein including alkyl atoms (in particular lower alkyl) to provide “alkylalkoxy” radicals; halo atoms, such as fluoro, chloro or bromo, to provide “haloalkoxy” radicals (e.g. fluoromethoxy, chloromethoxy, trifluoromethoxy, difluoromethoxy, trifluoroethoxy, fluoroethoxy, tetrafluoroethoxy, pentafluoroethoxy, and fluoropropoxy) and “haloalkoxyalkyl” radicals (e.g. fluoromethoxymethyl, chloromethoxyethyl, trifluoromethoxymethyl, difluoromethoxyethyl, and trifluoroethoxymethyl).

The term “acyl”, alone or in combination, means a carbonyl or thiocarbonyl group bonded to a radical selected from, for example, hydrogen, optionally substituted alkyl (e.g. haloalkyl), alkenyl, alkynyl, alkoxy (“acyloxy” including acetyloxy, butyryloxy, iso-valeryloxy, phenylacetyloxy, benzoyloxy, p-methoxybenzoyloxy, and substituted acyloxy such as alkoxyalkyl and haloalkoxy), halo, sulfinyl (e.g. alkylsulfinylalkyl), sulfonyl (e.g. alkylsulfonylalkyl), thioalkyl, amino (e.g., alkylamino or dialkylamino), and aralkoxy. Illustrative examples of “acyl” radicals are formyl, acetyl, 2-chloroacetyl, 2-bromacetyl and the like.

In aspects of the invention, “acyl” refers to a group —C(O)R⁶, where R⁶ is hydrogen or alkyl. Examples include, but are not limited to formyl, acetyl, and the like.

In aspects of the invention, the scyllo-inositol compound is a pure or substantially pure scyllo-inositol.

In aspects of the invention, a scyllo-inositol compound does not include a scyllo-inositol compound substituted with one or more phosphate group.

Particular embodiments of the invention utilize scyllo-inositol compounds of the formula Ia or Ib wherein one or more of the hydroxyl groups is replaced with alkyl, in particular C₁-C₄ alkyl, more particularly methyl or ethyl; acyl; chloro or fluoro; alkenyl; —NHR¹ wherein R¹ is hydrogen, acyl, alkyl or —R²R³ wherein R² and R³ are the same or different and represent acyl or alkyl; —SR⁴ wherein R⁴ is hydrogen, alkyl, or —O₃H; and —OR⁵ wherein R⁵ is hydrogen, alkyl, or —SO₃H, —SR⁴ wherein R⁴ is hydrogen, alkyl, or —O₃H; alkoxy, or —OR⁵ wherein R⁵ is —SO₃H.

Particular embodiments of the invention utilize scyllo-inositol compounds of the formula Ia or Ib wherein one or more of the hydroxyl groups is replaced with alkyl; substituted alkyl; acyl; alkenyl; substituted alkenyl; —NHR¹ wherein R¹ is hydrogen, acyl, alkyl, or —R²R³ wherein R² and R³ are the same or different and represent acyl or alkyl; —SR⁴ wherein R⁴ is hydrogen, alkyl, or —O₃H; alkoxy, or —OR⁵ wherein R⁵ is —SO₃H.

Particular embodiments of the invention utilize scyllo-inositol compounds of the formula Ia or Ib wherein one or more of the hydroxyl groups is replaced with alkyl; substituted alkyl; acyl; alkenyl; substituted alkenyl; alkynyl; substituted alkynyl; alkoxy; substituted alkoxy; halogen; thiol; —NHR¹ wherein R¹ is hydrogen, acyl, alkyl or —R²R³ wherein R² and R³ are the same or different and represent acyl or alkyl; —PO₃H₂; —SR⁴ wherein R⁴ is hydrogen, alkyl, or —O₃H; or —OR⁵ wherein R⁵ is —SO₃H.

Particular embodiments of the invention utilize scyllo-inositol compounds of the formula Ia or Ib wherein one or more of the hydroxyl groups is replaced with alkyl; substituted alkyl; acyl; alkenyl; substituted alkenyl; alkynyl; substituted alkynyl; alkoxy; substituted alkoxy; halogen; or thiol.

Particular embodiments of the invention utilize scyllo-inositol compounds of the formula Ia or Ib wherein one of the hydroxyl groups is replaced with alkyl, in particular C₁-C₄ alkyl, more particularly methyl.

Particular embodiments of the invention utilize scyllo-inositol compounds of the formula Ia or Ib wherein one of the hydroxyl groups is replaced with alkoxy, in particular C₁-C₄ alkoxy, more particularly methoxy or ethoxy, most particularly methoxy.

Particular embodiments of the invention utilize scyllo-inositol compounds of the formula Ia or Ib wherein one of the hydroxyl groups is replaced with halogen, in particular chloro or fluoro, more particularly fluoro.

Particular embodiments of the invention utilize scyllo-inositol compounds of the formula Ia or Ib wherein one of the hydroxyl groups is replaced with thiol.

In embodiments of the invention, the scyllo-inositol compound is O-methyl-scyllo-inositol

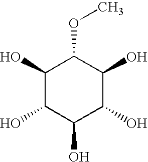

In embodiments of the invention, the scyllo-inositol compound is 1-chloro-1-deoxy-scyllo-inositol.

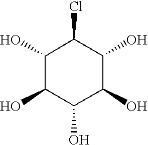

In embodiments of the invention, the scyllo-inositol compound designated AZD-103/ELND005 (Elan Corporation) is used in the medicaments, compositions, dosage forms, methods and uses disclosed herein.

A scyllo-inositol compound includes a functional derivative, a chemical derivative, or variant. A “functional derivative” refers to a compound that possesses an activity (either functional or structural) that is substantially similar to the activity of a scyllo-inositol compound disclosed herein. The term “chemical derivative” describes a molecule that contains additional chemical moieties which are not normally a part of the base molecule. The term “variant” is meant to refer to a molecule substantially similar in structure and function to a scyllo-inositol compound or a part thereof. A molecule is “substantially similar” to a scyllo-inositol compound if both molecules have substantially similar structures or if both molecules possess similar biological activity. The term “analog” includes a molecule substantially similar in function to a scyllo-inositol. An “analog” can include a chemical compound that is structurally similar to another but differs slightly in composition. Differences include without limitation the replacement of an atom or functional group with an atom or functional group of a different element. Analogs and derivatives may be identified using computational methods with commercially available computer modeling programs.

A scyllo-inositol compound includes a pharmaceutically functional derivative. A “pharmaceutically functional derivative” includes any pharmaceutically acceptable derivative of a scyllo-inositol compound, for example, an ester or an amide, which upon administration to a subject is capable of providing (directly or indirectly) a scyllo-inositol or an active metabolite or residue thereof. Such derivatives are recognizable to those skilled in the art, without undue experimentation (see for example Burger's Medicinal Chemistry and Drug Discovery, 5.sup.th Edition, Vol 1: Principles and Practice, which has illustrative pharmaceutically functional derivatives).

The scyllo-inositols used in the invention may be amorphous or may have different crystalline polymorphs, possibly existing in different solvation or hydration states. By varying the form of a drug, it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility. Examples of scyllo-inositol polymorphs which may be used in the present invention include the polymorphs described in Day, G M et al, Crystal Growth & Design, 6(10), 2006.

Solvates of scyllo-inositol compounds formed with water or common organic solvents are also intended to be encompassed within the invention. In addition, hydrate forms of the compounds and their salts are encompassed within this invention.

The term “solvate” means a physical association of a compound with one or more solvent molecules or a complex of variable stoichiometry formed by a solute and a solvent, for example, water, ethanol, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, the solvents selected do not interfere with the biological activity of the solute. Solvates encompass both solution-phase and isolatable solvates. Representative solvates include hydrates, ethanolates, methanolates, and the like. The term “hydrate” means a solvate wherein the solvent molecule(s) is/are H₂O, including, mono-, di-, and various poly-hydrates thereof. Solvates can be formed using various methods known in the art.

Crystalline scyllo-inositols can be in the form of a free base, a salt, or a co-crystal. Free base compounds can be crystallized in the presence of an appropriate solvent in order to form a solvate. Acid salt compounds (e.g. HCl, HBr, benzoic acid) can also be used in the preparation of solvates. For example, solvates can be formed by the use of acetic acid or ethyl acetate. The solvate molecules can form crystal structures via hydrogen bonding, van der Waals forces, or dispersion forces, or a combination of any two or all three forces. The amount of solvent used to make solvates can be determined by routine testing. For example, a monohydrate of a scyllo-inositol would have about 1 equivalent of solvent (H₂O) for each equivalent of a scyllo-inositol. However, more or less solvent may be used depending on the choice of solvate desired.

Prodrugs of scyllo-inositols compounds are encompassed within the term. The term “prodrug” means a covalently-bonded derivative or carrier of the parent compound or active drug substance which undergoes at least some biotransformation prior to exhibiting its pharmacological effect(s). In general, such prodrugs have metabolically cleavable groups and are rapidly transformed in vivo to yield the parent compound, for example, by hydrolysis in blood, and generally include esters and amide analogs of the parent compounds. A prodrug may be formulated to improve chemical stability, improve patient acceptance and compliance, improve bioavailability, prolong duration of action, improve organ selectivity, improve formulation (e.g., increased hydrosolubility), and/or decrease side effects (e.g., toxicity). In general, prodrugs themselves have weak or no biological activity and are stable under ordinary conditions. Prodrugs can be readily prepared from the parent compounds using methods known in the art, such as those described, for example, in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309 396; Burger's Medicinal Chemistry and Drug Discovery, 5th Ed., M. Wolff (ed.), John Wiley & Sons, 1995, particularly Vol. 1 and pp. 172 178 and pp. 949 982; Pro-Drugs as Novel Delivery Systems, T. Higuchi and V. Stella (eds.), Am. Chem. Soc., 1975; and Bioreversible Carriers in Drug Design, E. B. Roche (ed.), Elsevier, 1987. Examples of prodrugs include, but are not limited to esters (e.g., acetate, formate, and benzoate derivatives) and carbamates (e.g. N,N-dimethylaminocarbonyl) of hydroxy functional groups on scyllo-inositol compounds, and the like.

Scyllo-inositol compounds utilized in the invention may be prepared using reactions and methods generally known to the person of ordinary skill in the art, having regard to that knowledge and the disclosure of this application. The reactions are performed in a solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired scyllo-inositol compound. It will also be recognized that another major consideration in the development of a synthetic route is the selection of the protecting group used for protection of the reactive functional groups present in the scyllo-inositol compounds. An authoritative account describing the many alternatives to the skilled artisan is Greene and Wuts (Protective Groups In Organic Synthesis, Wiley and Sons, 1991).

The starting materials and reagents used in preparing scyllo-inositol compounds are either available from commercial suppliers such as the Aldrich Chemical Company (Milwaukee, Wis.), Bachem (Torrance, Calif.), Sigma (St. Louis, Mo.), or Lancaster Synthesis Inc. (Windham, N.H.) or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

The starting materials, intermediates, and scyllo-inositol compounds may be isolated and purified using conventional techniques, such as precipitation, filtration, distillation, crystallization, chromatography, and the like. The scyllo-inositol compounds may be characterized using conventional methods, including physical constants and spectroscopic methods, in particular HPLC.

Scyllo-inositol compounds which are basic in nature can form a wide variety of different salts with various inorganic and organic acids. In practice it is desirable to first isolate a scyllo-inositol compound from the reaction mixture as a pharmaceutically unacceptable salt and then convert the latter to the free base compound by treatment with an alkaline reagent and subsequently convert the free base to a pharmaceutically acceptable acid addition salt. The acid addition salts of the base compounds are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

Scyllo-inositol compounds which are acidic in nature are capable of forming base salts with various pharmacologically acceptable cations. These salts may be prepared by conventional techniques by treating the corresponding acidic compounds with an aqueous solution containing the desired pharmacologically acceptable cations and then evaporating the resulting solution to dryness, preferably under reduced pressure. Alternatively, they may be prepared by mixing lower alkanolic solutions of the acidic compounds and the desired alkali metal alkoxide together and then evaporating the resulting solution to dryness in the same manner as before. In either case, stoichiometric quantities of reagents are typically employed to ensure completeness of reaction and maximum product yields.

Scyllo-inositol compounds can be prepared using conventional processes or they may be obtained from commercial sources. For example, scyllo-inositol compounds can be prepared using chemical and/or microbial processes. In aspects of the invention, a scyllo-inositol compound is produced using process steps described by M. Sarmah and Shashidhar, M., Carbohydrate Research, 2003, 338, 999-1001; Husson, C., et al, Carbohyrate Research 307 (1998) 163-165; Anderson R. and E. S. Wallis, J. American Chemical Society (US), 1948, 70:2931-2935; Weissbach, A., J Org Chem (US), 1958, 23:329-330; Chung, S. K. et al., Bioorg Med. Chem. 1999, 7(11):2577-89; or Kiely D. E., and Fletcher, H. G., J. American Chemical Society (US) 1968, 90:3289-3290; described in JP09-140388, DE 3,405,663 (Merck Patent GMBH), JP04-126075, JP05-192163, or WO06109479, or described in WO05035774, US20060240534, EP1674578, and JP03-102492 (Hokko Chemical Industries). In particular aspects of the compositions and methods of the invention, a scyllo-inositol compound is prepared using the chemical process steps described in Husson, C., et al, Carbohydrate Research 307 (1998) 163-165. In other aspects of the compositions and methods of the invention, a scyllo-inositol compound is prepared using microbial process steps similar to those described in WO05035774 (EP1674578 and US20060240534) JP2003102492, or JP09140388 (Hokko Chemical Industries). Derivatives may be produced by introducing substituents into a scyllo-inositol compound using methods well known to a person of ordinary skill in the art.

The term “macular degeneration-related disorder” includes a condition which alters or damages the integrity of the macula (e.g., damage to the retinal pigment epithelium or Bruch's membrane) or in which the retinal macula degenerates or becomes dysfunctional. The retinal macula may degenerate or become dysfunctional as a result of one or more of the following events: a decreased growth of cells of the macula, increased death or rearrangement of cells of the macula such as retinal pigment epithelium cells, and loss of normal biological function. The condition can involve a loss of integrity of the histoarchitecture of the cells and/or extracellular matrix of the macula and/or the loss of function of macula cells. Examples of macular degeneration-related disorders include, without limitation, age-related macular degeneration, North Carolina macular dystrophy, Sorsby's fundus dystrophy, Stargardt's disease, pattern dystrophy, Best disease, dominant drusen, and malattia leventinese (radial drusen). The disorders also include extramacular changes that occur prior to, or following dysfunction and/or degeneration of the macula. By way of example, the disorders include retinal detachment, chorioretinal degenerations, retinal degenerations, mucopolysaccharidoses, photoreceptor degenerations, retinal pigment epithelium degenerations, rod-cone dystrophies, cone-rod dystrophies and cone degenerations.

In an embodiment, the macular degeneration-related disorder is age-related macular degeneration (AMD), both wet and dry forms, early or late stage, in an individual. In one specific embodiment, the disorder is associated with destruction and loss of the photoreceptors in the macula region of the retina resulting in decreased central vision and, in advanced cases, legal blindness.

In a particular embodiment, the macular degeneration-related disorder is the dry form of age-related macular degeneration. This phenotype is associated with cell death (e.g. loss of photoreceptors) within the light-sensitive macula which is required for fine vision. Drusen, a yellow deposit that accumulates between the RPE basal lamina and the inner collagenous layer of Bruch's membrane (van der Schaft et al, Opthalmol. 99:278-86, 1992' Mullins et al., in Degenerative retinal Disesaes (pp. 1-10). New York: Plenum Press, 1997), is a common early sign of dry AMD. A subject may be suffering from an early, intermediate or advanced stage of dry AMD, in one or both eyes. In aspects of the invention a subject is suffering from early age-related macular degeneration or age-related maculopathy. In aspects of the invention, a subject is suffering from pre-symptomatic age-related maculopathy.

In a particular embodiment, the macular degeneration-related disorder is neovascular, exudative or the wet form of age-related macular degeneration, in particular the classic or occult type (i.e., classic choroidal neovascularization and occult choroidal neovascularization). This phenotype is caused by growth of abnormal blood vessels under the macula which leak blood and fluid destroying the central retina resulting in the deterioration of sight.

In a particular embodiment of the invention the disorder is choroidal neovascularization secondary to age-related macular degeneration.

### Medicaments

A scyllo-inositol compound or salts thereof as an active ingredient can be directly administered to a patient, but it is preferably administered as a preparation in the form of a medicament or pharmaceutical compositions containing the active ingredient and pharmaceutically acceptable carriers, excipients, or vehicles. Therefore, the invention contemplates a medicament comprising an effective amount of an isolated, in particular pure, scyllo-inositol compound, for treating a macular degeneration-related disorder or symptoms caused by a macular degeneration-related disorder, suppressing the progression of a macular degeneration-related disorder, delaying diabilities associated with a macular degeneration-related disorder and/or providing therapeutic effects or prophylactic effects.

A medicament may comprise a scyllo-inositol compound in a therapeutically effective amount for modulating amyloid oligomerization and/or aggregation in ocular tissues, in particular macula or macular cells. In an aspect, the invention provides a medicament comprising a scyllo-inositol compound in a therapeutically effective amount for reducing and/or inhibiting amyloid oligomerization and/or aggregation in ocular tissues or dissolving and/or disrupting pre-existing amyloid oligomers or aggregates in ocular tissues. In an aspect, the invention provides a medicament comprising a scyllo-inositol compound in a therapeutically effective amount for reducing and/or inhibiting amyloid oligomerization and/or aggregation in macula or macular cells or dissolving and/or disrupting pre-existing amyloid oligomers or aggregates in macula or macular cells.

Medicaments or pharmaceutical compositions of the present invention or fractions thereof comprise suitable pharmaceutically acceptable carriers, excipients, and vehicles selected based on the intended form of administration, and consistent with conventional pharmaceutical practices. Suitable pharmaceutical carriers, excipients, and vehicles, formulations and techniques are described in the standard text, Remington: The Science and Practice of Pharmacy. (21st Edition, Popovich, N (eds), Advanced Concepts Institute, University of the Sciences in Philadelphia, Philadelphia, Pa. 2005). A scyllo-inositol compound can be formulated for a variety of modes of administration, including systemic and topical or local administration, in particular ocular administration. The medicaments may be formulated as sterile, substantially isotonic and in full compliance with all Good Manufacturing Practice (GMP) regulations of appropriate regulatory authorities such as the US Food and Drug Administration. A medicament or pharmaceutical composition of the invention can be in any form suitable for administration to a patient including a liquid solution (e.g. eye drops), suspension, emulsion, tablet, pill, capsule, sustained release formulation, or powder.

Examples of preparations which are appropriate for oral administration can include capsules, tablets, powders, fine granules, solutions and syrups, where the active components can be combined with an oral, non-toxic pharmaceutically acceptable inert carrier such as lactose, starch, sucrose, cellulose, methyl cellulose, magnesium stearate, glucose, calcium sulfate, dicalcium phosphate, sodium saccharine, magnesium carbonate, mannitol, sorbital, and the like. For oral administration in a liquid form, the active components may be combined with any oral, non-toxic, pharmaceutically acceptable inert carrier such as ethanol, glycerol, water, and the like. Suitable binders (e.g. gelatin, starch, corn sweeteners, natural sugars including glucose, natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the medicaments or components thereof. Medicaments as described herein can further comprise wetting or emulsifying agents, or pH buffering agents.

Medicaments which are appropriate for parenteral administration may include aqueous solutions, syrups, aqueous or oil suspensions and emulsions with edible oil such as cottonseed oil, coconut oil or peanut oil. In aspects of the invention, medicaments for parenteral administration include sterile aqueous or non-aqueous solvents, such as water, isotonic saline, isotonic glucose solution, buffer solution, or other solvents conveniently used for parenteral administration of therapeutically active agents. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone. A medicament intended for parenteral administration may also include conventional additives such as stabilizers, buffers, or preservatives, e.g. antioxidants such as methylhydroxybenzoate or similar additives.

Examples of additives for medicaments that can be used for injection or drip include a resolvent or a solubilizer that can compose an aqueous injection or an injection to be dissolved before use, such as distilled water for injection, physiological saline and propylene glycol, isotonizing agents such as glucose, sodium chloride, D-mannitol, and glycerine, and pH modifiers such as inorganic acid, organic acid, inorganic bases or organic base.

A scyllo-inositol compound may be admixed, encapsulated, conjugated or otherwise associated with molecules to facilitate uptake, distribution and/or absorption of the compound. Various known delivery systems can be used to administer a medicament of the invention, e.g. encapsulation in liposomes, microparticles, microcapsules, and the like. Medicaments can also be formulated as pharmaceutically acceptable salts as described herein.

In aspects, a medicament of the invention is a solution, suspension, or emulsion (dispersion) in a suitable ophthalmic formulation, and optionally comprising an appropriate buffer system (e.g., sodium phosphate, sodium acetate, sodium citrate, or sodium borate). Formulations for intraocular or periocular administration may additionally comprise physiologically balanced irrigating solutions which are adapted to maintain the physical structure and function of the tissue during invasive or noninvasive medical procedures. A physiologically balanced irrigating solution may generally comprise electrolytes (e.g., sodium, potassium, calcium, magnesium, and/or chloride); an energy source (e.g., dextrose); and a buffer to maintain the pH of the solution at or near physiological levels. Physiologically balanced intraocular solutions are well-known and/or commercially available and include Lactated Ringers Solution, BSS® Sterile Irrigating Solution, and BSS Plus® Intraocular Irrigating Solution (Alcon Laboratories, Inc. Fort Worth, Tex.).

In aspects, a medicament of the invention is an ophthalmic formulation, including a topical ophthalmic formulation. In a particular aspect, an ophthalmic formulation is provided comprising a scyllo-inositol compound and an opthalmologically acceptable carrier, excipient, or vehicle. An ophthalmic formulation may comprise opthalmologically acceptable preservatives, surfactants, viscosity enhancers, buffers, sodium chloride and/or water to form aqueous sterile ophthalmalic solutions and suspensions. An ophthalmic gel formulation is also contemplated comprising a scyllo-inositol compound and a hydrophilic base (e.g., derived from carboxyvinyl polymers such as Carbopol® (BF Goodrich Company)), and optionally preservatives and tonicity agents.

In aspects of the invention, a composition is provided that is suitable for topical administration. Such a medicament may comprise a liquid with a scyllo-inositol compound in solution, suspension or both. Liquid compositions comprise gels and/or are aqueous. A composition may comprise an ointment. In embodiments of the invention, the medicament is an in situ gellable aqueous composition, more particularly an in situ gellable aqueous solution. A composition may comprise an effective amount of a gelling agent to allow gelling upon contact with the eye or lacrimal fluid in the exterior of the eye. An aqueous composition may have an opthalmically compatible pH and osmolarity. The medicament may be in the form of an opthalmalic depot formulation for subconjunctival administration. The scyllo-inositol compound may be in the form of microparticles embedded in a biocompatible pharmaceutically acceptable polymer or lipid encapsulating agent. Depot formulations can be adapted to release the active scyllo-inositol compound over an extended period. A polymer or lipid agent matrix may be adapted to degrade sufficiently to enable transport from the site of administration after release of all or substantially all of a scyllo-inositol compound. Depot formulations may be liquid formulations with a pharmaceutically acceptable polymer and dispersed or dissolved scyllo-inositol compound. The polymer may form a depot for example by gelifying or precipitating at the injection site. A composition may comprise a solid article that can release a scyllo-inositol compound and is suitable for implantation in the eye. A solid article may comprise polymers and it can be bioerodible or non-bioerodible. The solid article may be implanted in a suitable location in the eye including between the eye and eyelid or in the conjunctival sac.

In embodiments, the invention provides a variety of medicaments or pharmaceutical compositions that facilitate targeting of a scyllo-inositol compound to a site in the eye. In certain embodiments, a composition is provided comprising a scyllo-inositol compound, and a moiety that binds to a component present in the eye of a subject at risk of or suffering from a macular degeneration-related disorder. In embodiments, the component is a cellular marker expressed on or at the surface of a cell such as an endothelial cell or retinal pigment epithelial cell. For example, the cellular markers are tissue factor (TF), CD68, claudin, the protein encoded by the RPE65 gene, CD45 and ICAM-1. In certain embodiments of the invention, the scyllo-inositol compound and moiety are linked. The linkage may be covalent or non-covalent and can be direct or indirect. A moiety may be an antibody or a ligand. A ligand may be a hormone, growth factor, oligo- or polysaccharide, aptamer or neurotransmitter that binds to particular receptors such as Factor VII, Factor Vila, or sialyl lewis^{X} (SLe^{X}).

A medicament can be sterilized by, for example, filtration through a bacteria retaining filter, addition of sterilizing agents to the medicament, irradiation of the medicament, or heating the medicament. Alternatively, the medicaments may be provided as sterile solid preparations e.g., lyophilized powder, which are readily dissolved in sterile solvent immediately prior to use.

After medicaments have been prepared, they can be placed in an appropriate container and labeled for treatment of an indicated condition (i.e., a macular degeneration-related disorder). For administration of a medicament, such labeling would include amount, frequency, and method of administration.

A scyllo-inositol compound may be in a form suitable for administration as a dietary supplement. A supplement may optionally include inactive ingredients such as diluents or fillers, viscosity-modifying agents, preservatives, flavorings, colorants, or other additives to conventional in the art. By way of example only, conventional ingredients such as beeswax, lecithin, gelatin, glycerin, caramel, and carmine may be included. A dietary supplement may be provided as a liquid dietary supplement (e.g., a dispensable liquid) or alternatively the compositions may be formulated as granules, capsules or suppositories. The liquid supplement may include a number of suitable carriers and additives including water, glycols, oils, alcohols, flavoring agents, preservatives, coloring agents and the like. In capsule, granule or suppository form, the dietary compositions are formulated in admixture with a pharmaceutically acceptable carrier. A supplement may be presented in the form of a softgel which is prepared using conventional methods. A softgel typically includes a layer of gelatin encapsulating a small quantity of the supplement. A supplement may also be in the form of a liquid-filled and sealed gelatin capsule, which may be made using conventional methods.

To prepare a dietary supplement composition in capsule, granule or suppository form, one or more compositions comprising scyllo-inositol compounds may be intimately admixed with a pharmaceutically acceptable carrier according to conventional formulation techniques. For solid oral preparations such as capsules and granules, suitable carriers and additives such as starches, sugars, diluents, granulating agents, lubricants, binders, disintegrating agents and the like may be included.

According to the invention, a kit is provided. In an aspect, the kit comprises a scyllo-inositol compound or a medicament described herein in kit form. In aspects of the invention, the kits may be useful for any of the methods disclosed herein, including, without limitation treating a subject suffering from a macular degeneration-related disorder. Kits of the invention may contain instructions for practicing any of the methods described herein. A medicament or formulation in a kit of the invention may comprise any of the ophthalmic formulations or compositions disclosed herein.

In embodiments of the invention, a kit of the invention comprises a container described herein and a second container comprising a buffer. A kit may additionally include other materials desirable from a commercial and user standpoint, including, without limitation, buffers, diluents, filters, needles, syringes, and package inserts with instructions for performing any methods disclosed herein.

The kit can be a package which houses a container which contains a scyllo-inositol compound or medicament of the invention and also houses instructions for administering the scyllo-inositol compound or medicament to a subject. The invention further relates to a commercial package comprising a scyllo-inositol compound or medicament together with instructions for its use. In particular, a label may include amount, frequency and method of administration.

In aspects of the invention, a pharmaceutical pack or kit is provided comprising one or more containers filled with one or more of the ingredients of a medicament of the invention to provide one or more therapeutic effects. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

In certain embodiments, a medicament, composition or formulation described herein can be a unit-of-use package which is a convenient, prescription size, patient ready unit labeled for direct distribution by health care providers. A unit-of-use package contains a medicament, composition or formulation in an amount necessary for a typical treatment interval and duration for a given indication. In an embodiment of the invention, a unit-of-use package is provided comprising, for example, a scyllo-inositol compound in an amount sufficient to treat an average sized adult male or female daily or once or twice weekly.

The invention also relates to articles of manufacture and kits containing materials useful for treating macular degeneration-related disorders. An article of manufacture may comprise a container with a label. Examples of suitable containers include bottles, vials, and test tubes which may be formed from a variety of materials including glass and plastic. A container holds a medicament or formulation comprising a scyllo-inositol compound which is effective for treating a macular degeneration-related disorder. The label on the container indicates that the medicament or formulation is used for treating macular degeneration-related disorders and may also indicate directions for use. The container may also be adapted for administration of the composition to the eye, such as a bottle for eye drops. A container or unit dosage may also be adapted for implantation or injection into the eye or tissues surrounding the eye such as the periocular tissue. In aspects of the invention, a medicament or formulation in a container may comprise any of the ophthalmic medicaments or formulations disclosed herein.

### Treatment Methods

The invention contemplates the use of effective amounts, in particular therapeutically effective amounts, of a scyllo-inositol compound or medicament of the invention for treating a macular degeneration-related disorder, in particular preventing, and/or ameliorating disease severity, disease symptoms, periodicity of recurrence of a macular degeneration-related disorder, and/or delaying disability associated with a macular degeneration-related disorder. In aspects, the invention contemplates treating in mammals a macular degeneration-related disorder using the medicaments or treatments described herein. Such uses and treatments may be effective for retarding the effects of a macular degeneration-related disorder, including specifically, but not exclusively, degeneration of ocular tissue and/or ocular function.

According to the invention, a scyllo-inositol compound may be administered to any subject in the general population as prophylaxis against the possibility that the person may in the future develop a macular degeneration-related disorder. In particular embodiments, a scyllo-inositol compound may be administered to a subject suspected of being at risk for a macular degeneration-related disorder, for example, by virtue of being in a family with a higher than normal incidence of a macular degeneration-related disorder or due to a defined genetic proclivity. Another category of subjects who may, in particular embodiments of the invention be prophylactically treated with a scyllo-inositol compound, are persons who have experienced an environmental exposure believed to be associated with the development of a macular degeneration-related disorder such as exposure to pesticides, herbicides, organic solvents, mercury, lead, etc.

In an aspect, the invention provides use of a scyllo-inositol compound or medicament of the invention to prophylactically treat persons in the general population and more particularly persons believed to be at risk for developing a macular degeneration-related disorder because of, for example, a positive family history for the disease and/or the presence of a genetic defect. In addition, a scyllo-inositol compound or a medicament of the invention may be used to treat persons already diagnosed with a macular degeneration-related disorder (e.g. AMD) to delay the progression of existing ocular impairment or disabilities and/or to delay the onset of not yet detected ocular impairment or disabilities.

In addition, a scyllo-inositol compound may be administered to a subject in the early stages of a macular degeneration-related disorder or age-related maculopathy, in particular upon a determination that the diagnosis of a macular degeneration-related disorder is probable. A period considered an “early stage” can be the first month or 2, 3, 6, 8, or 12 months after the onset of symptoms or diagnosis. A subject may be pre-symptomatic in the early stages of a macular degeneration-related disorder or age-related maculopathy.

In aspects of the invention, a scyllo-inositol compound may be administered to a subject in the later stages of a disorder to delay the onset of symptoms or diagnosis. A period considered a “later stage” can be more than 6, 8, 12, 18 or 24 months after the onset of symptoms or diagnosis.

The medicaments and treatments of the invention are selected to provide therapeutic effects and/or prophylactic effects. In an embodiment, therapeutic and/or prophylactic effects of a medicament or treatment of the invention for a macular degeneration related-disorder can manifest as one or more or all of the following:
a) A reduction, slowing or prevention of an increase in, or an absence of symptoms of a macular degeneration-related disorder after administration to a subject with symptoms of the disorder.
b) A slowing or arrest of the progress of a macular degeneration-related disorder.
c) A modulation of complement factors such as H, D, C3, Ba, C3d and/or C3b.
d) A delay in the progression of existing ocular impairment and/or a delay of the onset of not yet detected ocular impairment.
e) A reduction, slowing or prevention of an increase in degeneration of ocular tissue relative to the levels measured in the absence of a scyllo-inositol compound or medicament disclosed herein in subjects with symptoms of a macular degeneration-related disorder, in particular age-related macular degeneration. In aspects of the invention, a scyllo-inositol compound or medicament induces at least about a 2%, 5%, 10%, 15%, 20%, 30%, 40%, 50%, 60%, 70%, 80%, or 90% decrease in degeneration of ocular tissue.
f) An increase or restoration of ocular function after administration to a subject with symptoms of a macular degeneration-related disorder. In aspects of the invention a scyllo-inositol compound or medicament disclosed herein induces at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% increase in ocular function in a subject.
g) An improvement in visual acuity. In particular an increase in mean visual acuity in subjects, in particular an increase in mean visual acuity of at least about 1.2, 1.5, 1.75, 2, 3, 4, 5, 7, 8, 9, or 10 times compared with subjects not receiving a treatment in accordance with the invention.
h) A reduction or slowing of the rate of disease progression in a subject with a macular degeneration-related disorder.
i) A reduction, slowing or prevention of ocular dysfunction. In embodiments of the invention, a scyllo-inositol compound or medicament induces at least about a 2%, 5%, 10%, 15%, 20%, 30%, 40%, 50%, 60%, 70%, 80%, or 90% reduction or slowing of ocular dysfunction.
j) An improvement in any quantitative conditions of a subject's eye. In embodiments, the improvement may be an increase of at least 20%, 30%, 40%, 50%, 60%, 70%, 80%, 90%, 100%, 200% or 300% or more, in any quantitative measure of the condition of the subject's eye. In embodiments, the improvement may be a decrease of at least 20%, 30%, 40%, 50%, 60%, 70%, 80%, 90%, 100%, 200% or 300% or more, in any quantitative measure of the condition of the subject's eye
k) A reduction or inhibition of VEGF or VEGF activity. In embodiments of the invention, a scyllo-inositol compound or medicament induces at least about a 1%, 1.5%, 2%, 5%, 10%, 15%, 20%, 30%, 40%, 50%, 60%, 70%, 80%, or 90% reduction in VEGF or VEGF activity.
l) A reduction of existing or alternative treatments in a subject with a macular degeneration-related disorder. In embodiments, the therapeutic effect comprises a reduction in the number of administrations of an existing therapy, more particularly a reduction in the number of injections of Lucentis.
m) A reduction of side-effects or adverse events of existing or alternative treatments in a subject with a macular degeneration-related disorder. Examples of side-effects and adverse events include without limitation endophthalmitis, retinal detachment, iatrogenic treatment cataract, anterior chamber inflammation, blurred vision, cataract, conjunctival hemorrhage, corneal edema, conjunctival edema, corneal abrasion, corneal deposits, corneal epithelium disorder, eye discharge, eye irritation, eye pain, hypertension, increased intraocular pressure (IOP), foreign body sensation in eyes, increased lacrimation, eye puritis, visual disturbance, blepharitis, subretinal fibrosis, ocular hyperemia, maculopathy, ocular discomfort, increase in intraocular pressure, vitreous detachment, punctuate keratitis, reduced visual acuity, visual disturbance, vitreous floaters, vitreous opacities, blepharitis, conjunctivitis, allergic conjunctivitis, photopsia, vitreous disorder, intraocular inflammation, eye inflammation, conjunctival hyperemia, posterior capsule opacification, retinal exudates, detachment of the retinal pigment epithelium, dry eye, eye swelling, eyelid irritation, meibomianitis, mydriasis, periorbital hematoma, retinal edema, retinal hemorrhage, and vitreous hemorrhage.
n) An increase in survival or longevity in a subject with symptoms of a macular degeneration-related disorder.
o) A reduction in the kinetics of assembly of oligomers and/or aggregates comprising amyloid, in particular a 2%, 5%, 10%, 15%, 20%, 30%, 40%, 50%, 60%, 70%, 80%, or 90% reduction in the kinetics of assembly of such oligomer and/or aggregates.
p) A reduction, slowing or prevention of an increase in accumulation of amyloid, or oligomers or aggregates comprising amyloid in ocular tissue relative to the levels measured in the absence of a scyllo-inositol compound or medicament disclosed herein in subjects with symptoms of a macular degeneration related-disorder. In embodiments of the invention, the scyllo-inositol compound or medicament induces at least about a 2%, 5%, 10%, 15%, 20%, 30%, 40%, 50%, 60%, 70%, 80%, or 90% decrease in accumulation of amyloid, or oligomers or aggregates comprising amyloid.
q) A prevention, reduction or inhibition of amyloid β aggregation or assembly of oligomers or aggregates comprising amyloid β (e.g., drusen) in ocular tissues.
r) A reversal or reduction of amyloid β or oligomers or aggregates comprising amyloid β after the onset of symptoms of a macular degeneration-related disorder.
s) The dissolution and/or disruption of amyloid β, or oligomers or aggregates comprising amyloid β in ocular tissues.
t) The enhanced clearance of amyloid β, or oligomers or aggregates comprising amyloid β in ocular tissues.

In aspects of the invention the therapeutic or prophylactic effects of a medicament or treatment of the invention can manifest as (a) and (b); (a), (b) and (c); (a), (b), (c) and (d); (a), (b), (c), (d), (e) and (f); (a), (b), (c), (d), (e), (f) and (g); (a), (b), (c), (d), (e), (f), (g) and (h); (a), (b), (c), (d), (e), (f), (g), (h) and (i); (e), (f), (g), (i), (k) and (l); (a), (b), (c), (d), (e), (f), (g), (h), (i) and (j); (a) to (k); (a) to (l); (a) to (m); (a) to (n); (a) to (o); (a) to (p); (a) to (q); (a) to (r); (a) to (s), or (a) to (t).

Scyllo-inositol compounds, medicaments and methods of the invention can be selected that have sustained therapeutic effects, and in certain embodiments statistically significant sustained therapeutics effects. The therapeutic effects may be sustained over several days, weeks, months or years thereby having a major beneficial impact on the severity of the disease and its complications. In aspects of the invention, a therapeutic effect may be sustained for a prolonged period of at least about 2 to 4 weeks, 2 to 5 weeks, 3 to 5 weeks, 2 to 6 weeks, 2 to 8 weeks, 2 to 10 weeks, 2 to 12 weeks, 2 to 14 weeks, 2 to 16 weeks, 2 to 20 weeks, 2 to 24 weeks, 2 weeks to 12 months, 2 weeks to 18 months, 2 weeks to 24 months, or several years following treatment. The period of time a therapeutic effect is sustained may correlate with the duration and timing of the treatment. A subject may be treated continuously for about or at least about 1 week, 2 to 4 weeks, 2 to 6 weeks, 2 to 8 weeks, 2 to 10 weeks, 2 to 12 weeks, 2 to 14 weeks, 2 to 16 weeks, 2 weeks to 6 months, 2 weeks to 12 months, 2 weeks to 18 months, 2 weeks to 24 months, more than 24 months or several years, periodically or continuously.

The therapeutic effect may be a statistically significant effect in terms of statistical analysis of an effect of a scyllo-inositol compound, versus the effects without such a compound. “Statistically significant” or “significantly different” effects or levels may represent levels that are higher or lower than a standard. In embodiments of the invention, the difference may be 1.5, 2, 3, 4, 5, 6, 7, 8, 9, 10, 15, 20, 25, 50, 1-10, 1-20, 1-30 or 1-50 times higher or lower compared with the effect obtained without the compound.

Greater efficacy and potency of a treatment of the invention in some aspects may improve the therapeutic ratio of treatment, reducing untoward side effects and toxicity. It may also reduce or eliminate side effects of alternate treatments for a macular degeneration-related disorder. Selected methods of the invention may also improve long-standing disease even when treatment is begun long after the appearance of symptoms. In some embodiments, prolonged efficacious treatment can be achieved in accordance with the invention following administration of a scyllo-inositol compound or medicament comprising same.

The invention provides a method for treating a macular degeneration-related disorder in a subject comprising administering to the subject an effective amount of a scyllo-inositol compound.

In embodiments, the invention relates to a method for treating a macular degeneration-related disorder comprising contacting amyloid oligomers or aggregates in the retina, in particular macula, in a subject with a therapeutically effective amount of a scyllo-inositol compound or a medicament of the invention.

In embodiments, the invention provides a method for treating a macular degeneration-related disorder by providing a medicament comprising a scyllo-inositol compound in an effective amount to disrupt amyloid oligomers and/or aggregates for a prolonged period following administration.

In embodiments, the invention provides a method involving administering to a subject a therapeutically effective amount of a scyllo-inositol compound, a pharmaceutically acceptable salt thereof, or a medicament comprising a scyllo-inositol compound and a pharmaceutically acceptable carrier, excipient, or vehicle which modulates folding, oligomerization and/or aggregation of amyloid in ocular tissue.

In a further embodiment, the invention provides a method involving administering to a subject a therapeutically effective amount of a scyllo-inositol compound, a pharmaceutically acceptable salt thereof, or a medicament comprising a scyllo-inositol compound and a pharmaceutically acceptable carrier, excipient, or vehicle which causes dissolution/disruption of pre-existing amyloid, amyloid oligomers or aggregates in ocular cells or tissues.

In an embodiment, the invention provides a method for preventing or inhibiting assembly or slowing deposition of amyloid in ocular tissue comprising administering an effective amount for preventing or inhibiting assembly or slowing deposition of amyloid or oligomers or aggregates comprising amyloid in ocular cells, of a scyllo-inositol compound, a pharmaceutically acceptable salt thereof, or a medicament comprising a scyllo-inositol compound and a pharmaceutically acceptable carrier, excipient, or vehicle.

In an embodiment, the invention provides a method of reversing or reducing amyloid or oligomers and/or aggregates comprising amyloid in ocular cells after the onset of symptoms of a macular degeneration-related disorder in a subject comprising administering to the subject a therapeutically effective amount of a scyllo-inositol compound, a pharmaceutically acceptable salt thereof, or a medicament comprising a scyllo-inositol compound and a pharmaceutically acceptable carrier, excipient, or vehicle.

In an aspect, the invention provides a method for enhancing clearance of amyloid or oligomers or aggregates comprising amyloid in ocular cells in a subject comprising administering a therapeutically effective amount for enhancing clearance of amyloid or oligomers or aggregates comprising amyloid in ocular cells, of a scyllo-inositol compound, a pharmaceutically acceptable salt thereof, or a medicament comprising a scyllo-inositol compound and a pharmaceutically acceptable carrier, excipient, or vehicle.

In another aspect, the invention provides a method for treating a macular degeneration-related disorder comprising administering (e.g., intraocularly), an amount of a scyllo-inositol compound to a mammal, to reduce accumulation of amyloid and/or amyloid oligmers and/or aggregates in ocular cells for a prolonged period following administration.

In another aspect, the invention provides a method for preventing and/or treating a macular degeneration-related disorder, the method comprising administering to a mammal in need thereof a medicament comprising a scyllo-inositol compound in an effective amount to disrupt oligomerized and/or aggregated amyloid in ocular tissue following administration; and determining the amount of oligomerized and/or aggregated amyloid, thereby treating the disorder. The amount of oligomerized and/or aggregated amyloid may be measured using an antibody specific for amyloid or a scyllo-inositol compound labeled with a detectable substance.

In a further aspect, the invention provides a method for treating a macular degeneration-related disorder in a patient in need thereof which includes administering to the individual a medicament that provides a scyllo-inositol compound in a dose sufficient or effective to increase ocular function.

The invention in an embodiment provides a method for treating a macular degeneration-related disorder, the method comprising administering to a mammal in need thereof a medicament comprising a scyllo-inositol compound in an effective amount to reduce ocular dysfunction for a prolonged period following administration, thereby treating the ocular disease.

A method is provided for treating a subject with a macular degeneration-related disorder, comprising administering to the subject a therapeutically effective amount of a scyllo-inositol compound, wherein the subject has failed to respond to previous treatment with conventional therapeutic agents or procedures, thereby treating the subject. In an aspect, a method is provided for treating a subject with age-related macular degeneration who has failed to respond to conventional treatments comprising administering to the subject a therapeutically effective amount of a scyllo-inositol compound. In an aspect, a method is provided for treating a subject with age-related macular degeneration, comprising administering to the subject a therapeutically effective amount of a scyllo-inositol compound, wherein the subject has failed to respond to previous treatment with conventional therapeutic agents or procedures, thereby treating the subject.

The invention provides a method of treating a macular degeneration-related disorder in a subject in need thereof comprising administering a composition comprising or consisting essentially of a scyllo-inositol compound in a pharmaceutically acceptable formulation, and in an amount effective to treat a macular degeneration-related disorder without substantial toxicity to the patient. In an aspect, the invention provides a method of treating a macular degeneration-related disorder in a subject in need thereof comprising intraocularly injecting a composition comprising or consisting essentially of a scyllo-inositol compound in a pharmaceutically acceptable formulation and in an amount effective to treat a macular degeneration-related disorder without substantial toxicity to the subject. In an embodiment, a method is provided for treating a subject with age-related macular degeneration (AMD), comprising intraocularly injecting a composition comprising or consisting essentially of a scyllo-inositol compound in a pharmaceutically acceptable formulation and in an amount effective to treat AMD without substantial toxicity to the patient.

In an aspect of the invention, a human subject with macular degeneration-related disorder in one or both eyes is treated with a scyllo-inositol compound via intravitreal injection. In embodiments of the invention the subject is receiving conventional therapy, in particular Macugen or Lucentis.

A subject treated with a method of the invention may be monitored using methods known in the art, including without limitation, indirect opthalmoscopy, fundus photography, fluorescein angiography, optical tomography (OCT), electroretinography, external eye examination, slit lamp biomicroscopy, applanation tonometry, pachymetry and/or autorefaction.

The invention also contemplates the use of a medicament comprising at least one scyllo-inositol compound for treating a macular degeneration-related disorder or for the preparation of a medicament in treating a macular degeneration-related disorder. In an embodiment, the invention relates to the use of a therapeutically effective amount of at least one scyllo-inositol compound for providing therapeutic effects in treating a macular degeneration-related disorder or for the preparation of a medicament for providing therapeutic effects in treating a macular degeneration-related disorder. In another embodiment, the invention relates to the use of a prophylactically effective amount of at least one scyllo-inositol compound for preventing a macular degeneration-related disorder or symptoms thereof, or for the preparation of a medicament for preventing a macular degeneration-related disorder or symptoms thereof. In a still further embodiment the invention provides the use of a scyllo-inositol compound for prolonged or sustained treatment of a macular degeneration-related disorder or for the preparation of a medicament for prolonged or sustained treatment of a macular degeneration-related disorder. A prolonged or sustained treatment may be for a period of at least 4 weeks, 5 weeks, 6 weeks, 8 weeks, 10 weeks, 12 weeks, 14 weeks, 16 weeks, 20 weeks, 24 weeks, 30 weeks, 40 weeks, 52 weeks, or 78 weeks, more particularly about, 2 to 4 weeks, 2 to 5 weeks, 3 to 5 weeks, 2 to 6 weeks, 2 to 8 weeks, 2 to 10 weeks, 2 to 12 weeks, 2 to 16 weeks, 2 to 20 weeks, 2 to 24 weeks, 2 weeks to 12 months, 2 weeks to 24 months, 2 to 12 months, 2 to 14 months, 2 to 18 months, 3 to 12 months, 3 to 14 months, 3 to 18 months, 6 to 12 months, 6 to 14 months, 6 to 18 months or 6 to 24 months.

The present invention also includes compositions comprising or methods using a scyllo-inositol compound or medicaments/compositions of the invention in combination with one or more additional therapeutic agents or methods, in particular conventional therapeutic agents or procedures. In aspects of the invention, a subject may also receive photocoagulation therapy or photodynamic therapy (see for example, U.S. Pat. Nos. 5,756,541, 5,910,510, 6,599,891, 7,060,695, 7,015,240, US Published Applications Nos. 20030087889 and 20040019032). For example, a subject may receive photodynamic therapy that uses verteporfin as the photosensitizer (e.g. Visudyne Photodynamic Therapy (Novartis)). A patient may receive macular translocation surgery or may be treated using rheophoresis or laser surgery. Carotenoids, such as lutein and zeaxanthin, which are potent antioxidants found in high concentrations in the macular retina may also be administered to a subject [See, for example, Chopdar et al., BMJ 326, 485 (2003)]. A subject may also receive anti-vascular endothelial growth factor (anti-VEGF) therapeutics in combination with a scyllo-inositol compound. An anti-VEGF therapeutic may be any molecule that, directly or indirectly, binds to VEGF or down-regulates VEGF. Examples of anti-VEGF therapeutics include without limitation pegaptanib (Macugen®), ranibizumab (Lucentis®), bevacizumab (Avastin®), VEGF trap (e.g., aflibercept, VEGF Trap-Eye), or siRNA molecules Cand5 (Acuity Pharmaceuticals, Inc.) and Sirna-027 (Sirna Therapeutics). In some aspects, Triamcinolone (Kenalog) may be administered in combination with a scyllo-inositol compound. Other therapeutics that may be administered in combination with a scyllo-inositol compound are combretastatin A4 phosphate (CA4P) (ZYBRESTAT®), TG100801 (eye drop, TargeGen, Inc.), ATG3 (mecamylamine) (CoMentis, Inc.), and Othera (OT)-551 antioxidant eye drop (Othera Pharmaceuticals). Scyllo-inositol may be administered in combination with supplements such as one or more macular xanthophylls (lutein and zeaxanthin), long-chain omega-3 fatty acids (docosahexaenoic acid) [DHA], eicosapentaenoic acid [EPA] and zinc.

A method is provided for prolonging in a subject efficacy of a conventional therapy for treating a macular degeneration-related disorder (e.g. AMD) comprising administering to the subject receiving the conventional therapy a therapeutically effective amount of a scyllo-inositol compound. In embodiments, a therapeutically effective amount is administered to prolong the efficacy of the conventional therapy, increase time to relapse and/or reduce or eliminate side-effects or adverse effects of the therapy. In an aspect, the subject suffers from AMD. In a particular aspect the subject is receiving an anti-VEGF therapeutic, in particular Lucentis or Macugen.

A second therapy or agent and scyllo-inositol compound may be administered simultaneously or sequentially, in any order and for any period of time. Each component or therapy may be administered separately, but sufficiently close in time to provide the desired effect, in particular a therapeutic effect, more particularly a synergistic effect. The first compound or treatment may be administered in a regimen that additionally comprises treatment with the second compound or treatment. The active agents may be combined in one formulation or they may be in separate formulations. In an aspect, the scyllo-inositol compound is administered (e.g. for a period of time or continuously) following completion of the conventional therapy.

In an embodiment, a subject with wet AMD treated with an anti-VEGF treatment (e.g. Lucentis or Macugen) (preferably treated monthly for 3 months) is administered a scyllo-inositol compound or medicament/composition of the invention following interruption or cessation of the anti-VEGF treatment, in particular after relapse.

A subject selected for a treatment of the invention may have one or more of the following:
i. Visual acuity loss of greater than 5 letters with OCT evidence of fluid in the macula.
ii. Increase in OCT central retinal thickness of greater than 100 mM.
iii. New macular hemorrhage.
iv. New area of classic choroidal neovascularization.
v. Persistent fluid (by OCT) greater than one month after previous administration of the anti-VEGF therapeutic.

In an embodiment, a subject with wet AMD treated with an anti-VEGF treatment (e.g. Lucentis) is administered a scyllo-inositol compound or medicament of the invention following interruption or cessation of the anti-VEGF treatment, in particular after relapse.

The present invention provides methods to enhance or potentiate the effects of a conventional macular degeneration therapy and methods of treating a macular degeneration-related disorder in a subject by administering a therapeutically effective amount of a scyllo-inositol compound and optionally an anti-vascular endothelial growth factor (anti-VEGF) therapeutic, an anti-oxidant, photocoagulation therapy or photodynamic therapy, or alternatively a composition comprising a scyllo-inositol and an anti-vascular endothelial growth factor (anti-VEGF) therapeutic or an anti-oxidant.

A scyllo-inositol compound can be administered simultaneously, separately or in combination with a conventional macular degeneration therapy, under different dose and route regimens, to enhance the efficacy of the macular degeneration therapy in the treatment of a macular degeneration-related disorder in a subject compared to when such therapies are administered alone. Greater efficacy and/or potency of a treatment of the invention potentially improves the therapeutic ratio of treatment, reducing untoward side effects and toxicity. The methods of the invention may also enhance utility, improving long-standing treatment of a disorder.

A method of treatment of the invention may involve administration of a composition including a scyllo-inositol and an anti-vascular endothelial growth factor (anti-VEGF) therapeutic or an anti-oxidant. An alternate method of treatment includes the step of the administration of a composition comprising a scyllo-inositol followed by the step of the administration of a second pharmaceutical composition comprising an anti-vascular endothelial growth factor (anti-VEGF) therapeutic or an anti-oxidant. The administration of the scyllo-inositol compound can follow administration of the anti-vascular endothelial growth factor (anti-VEGF) therapeutic or an anti-oxidant. The administration of the pharmaceutical compositions can occur separately or simultaneously.

The invention provides a kit comprising as a first component a therapeutically effective amount of a sterile scyllo-inositol compound and as a second component a therapeutically effective amount of a sterile anti-vascular endothelial growth factor (anti-VEGF) therapeutic or an anti-oxidant for administration separately or in combination to a subject. The first and second component may be included in a single container. The components may be in sterile aqueous buffer or in the form of dry lyophilized powder or water free concentrate. Where the components are in the form of dry lyophilized powder the kit may further comprise sterile water for reconstituting the components. A kit may further comprise a notice in the form prescribed by a governmental agency regulating the manufacture, use or sale of pharmaceuticals or biological products which notice reflects approval by the agency for manufacture, use or sale of the kit for human administration, in particular to treat a macular degeneration related disorder. The invention also provides a kit for preparing a pharmaceutical composition for administration to a patient for treatment of a macular degeneration related disorder comprising a container comprising a therapeutically effective amount of a sterile scyllo-inositol compound and a therapeutically effective amount of a sterile anti-vascular endothelial growth factor (anti-VEGF) therapeutic or an anti-oxidant, and one or more pharmaceutically acceptable carrier or excipient capable of forming said pharmaceutical composition.

Therapeutic efficacy and toxicity of medicaments and methods of the invention may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Medicaments which exhibit large therapeutic indices are preferred.

Efficacy of a composition or method of the invention may be determined by various endpoints generally used in evaluating intraocular neovascular diseases. Examples of such endpoints include measuring one or more of the following:
a) vision loss, which can be assessed, for example, by measuring the mean change in best correction visual acuity (BCVA) from baseline to a desired time point (e.g., using the Early Treatment Diabetic Retinopathy Study testing protocol; Cotter S A, et al. Am J Opthalmol. 2003; 136:655-661);
b) the proportion of subjects who lose fewer than 15 letters in visual acuity at a desired time point compared to baseline;
c) the proportion of subjects who gain greater than or equal to 15 letters in visual acuity at a desired time point compared to baseline;
d) the proportion of subjects with a visual-acuity Snellen equivalent of 20/2000 or worse at a desired time point;
e) the NEI Visual Functioning Questionnaire;
f) intraocular pressure;
g) slitlamp pressure, and
h) the size of Choroidal Neovascularization (CNV) and amount of leakage of CNV at a desired time point using for example fluorescein angiography.
   Efficacy can also be assessed by performing ocular assessments such as performing an eye exam, assessing visual acuity, and assessing intraocular inflammation, and the like.

### Administration

Scyllo-inositol compounds and medicaments comprising or consisting of same can be administered by any means that produce contact of the active agent(s) with the agent's sites of action in the body of a subject or patient to produce a therapeutic or prophylactic effect, in particular a therapeutic effect. Methods of administration include without limitation, systemic, transpleural, intravenous, oral, intraarterial, intramuscular, topical, via inhalation (e.g., as mists or sprays), via nasal mucosa, subcutaneous, transdermal, intraperitoneal, gastrointestinal, and directly to the eye or tissues surrounding the eye. The scyllo-inositol compounds may be administered in the form of tablets, pills, powders, capsules, granules, injectables, creams, solutions, suppositories, emulsions, dispersions, and in other suitable forms. The compounds can be administered in liposome formulations. The scyllo-inositol compounds can also be administered as prodrugs.

In aspects of the invention, scyllo-inositol compounds or medicaments are administered to the eye or tissues associated with the eye. The compounds and medicaments may be administered topically to the eye and may be in the form of eye drops or eye washes. Intraocular administration of therapeutics intended for treatment of macular degeneration disorders are known in the art (see, for example, U.S. Pat. Nos. 5,632,984, 5,770,589 and 6,378,5260). The compounds and medicaments may also be administered by injection to the eye (intraocular injection) or to the tissues associated with the eye. They may also be administered by subconjunctival injection, trans-septal injection, intravitreal injection, transpleural injection, subretinal injection, periocular injection, sub-Tenon's injection, or retrobulbar injection. The scyllo-inositol compounds and medicaments may also be administered to a subject as an implant which is preferably a biocompatible and/or biodegradable sustained release formulation which gradually releases the compounds over a dosage period. Implants for ocular administration are well-known in the art; see for example, U.S. Pat. Nos. 5,501,856, 5,476,511 and 6,331,313. Scyllo-inositol compounds may also be administered using iontophoresis, for example using the methods described in U.S. Pat. No. 4,454,151, and US Patent Application Publication Nos. 20030181531 and 20040058313. In embodiments, the method of administration is intraocular and includes transretinal, subconjunctival bulbar, scleral pocket or scleral cutdown injection. In embodiments, the method of administration includes choroidal injection, transscleral injection, placing a scleral patch, and selective arterial catheterization.

A scyllo-inositol compound and medicament of the invention can be formulated for sustained release, for delivery locally or systemically. It lies within the capability of a skilled physician or veterinarian to select a form and route of administration that optimizes the effects of the medicaments and treatments to provide therapeutic effects.

A dosage regimen of the invention will vary depending upon known factors such as the pharmacodynamic characteristics of the selected scyllo-inositol compounds and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect.

An amount of a scyllo-inositol compound which will be effective in the treatment of a macular degeneration-related disorder to provide effects, in particular therapeutic effects, can be determined by standard clinical techniques. The precise dose to be employed in the formulation will also depend on the route of administration, and the seriousness of the disease, and will be decided according to the judgment of the practitioner and each patient's circumstances.

Suitable dosage ranges for administration are particularly selected to provide therapeutic effects. A pharmaceutical unit dosage of a scyllo-inositol compound is preferably fabricated and administered to provide a defined final concentration of the drug either in the blood, or in tissues of the eye and/or tissues associated with the eye.

A dosage range is generally effective for triggering the desired biological responses or therapeutic effects. The dosage ranges may generally be any of about 0.001 μg to about 5 g per kg per day, about 0.01 μg to about 5 g per kg per day, about 0.1 μg to about 5 g per kg per day, about 0.1 mg to about 5 g per kg per day, about 0.1 mg to about 2 g per kg per day, about 0.5 mg to about 5 g per kg per day, about 1 mg to about 5 g per kg per day, about 1 mg to about 500 mg per kg per day, about 1 mg to about 200 mg per kg per day, about 1 mg to about 100 mg per kg per day, about 5 mg to about 100 mg per kg per day, about 10 mg to about 100 mg per kg, about 25 mg to about 75 mg per kg per day, about 1 mg to about 50 mg per kg per day, about 2 mg to about 50 mg/kg/day, about 2 mg to about 40 mg per kg per day, or about 3 mg to about 25 mg per kg per day. In aspects of the invention, the dosage ranges are generally any of about 0.01 μg to about 2 g per kg, about 1 μg to about 2 g per kg, about 1 mg to about 2 g per kg, 5 mg to about 2 g per kg, about 1 mg to about 1 g per kg, about 1 mg to about 200 mg per kg, about 1 mg to about 100 mg per kg, about 1 mg to about 50 mg per kg, about 10 mg to about 100 mg per kg, or about 25 mg to 75 mg per kg of the weight of a subject. A medicament or scyllo-inositol compound may be administered once, twice or more daily, in particular once daily.

In some aspects of the invention, the dosages are administered directly to the eye in intravitreal injection, subconjunctival implants, or ophthalmic drop formulations. In intravitreal dosage forms, the dosage ranges are about any of 0.01 μg to about 10 mg, 0.01 μg to about 5 mg, 0.01 μg to about 1 mg, 0.01 μg to about 750 μg, 0.01 μg to about 500 μg, 0.1 μg to 10 mg, 0.1 μg to 5 mg, 0.1 μg to 1 mg, 0.1 μg to 750 μg, 0.1 μg to 500 μg, 1 μg to 10 mg, 1 μg to 5 mg, 1 μg to 1 mg, 1 μg to 750 μg or 1 μg to 500 μg per administration. In subconjunctivally dosage forms or drops, the dosage ranges are about any of 0.01 μg to about 20 mg, 0.01 μg to about 15 mg, 0.01 μg to about 10 mg, 0.01 μg to about 5 mg, 0.01 μg to about 1 mg, 0.1 μg to about 20 mg, 0.1 μg to 15 mg, 0.1 μg to 10 mg, 0.1 μg to 5 mg, 0.1 μg to 1 mg, 1 μg to 20 mg, 1 μg to 15 mg, 1 μg to 10 mg, 1 μg to 5 mg, 1 μg to 1 mg or 1 μg to 500 μg per administration. In the case of ophthalmic drops, the dosages may be administered once, twice or three-times daily. Preferred dosing intervals for injections and implants are weekly, biweekly, monthly, bi-monthly or longer.

In some aspects of the invention, the oral dosage ranges of a compound disclosed herein, administered once twice, three times or more daily, in particular once or twice daily, more particularly once daily, are about any of 0.01 μg to 5 g/kg, 1 μg to 2 g/kg, 1 to 5 g/kg, 1 to 3 g/kg, 1 to 2 g/kg, 1 to 1 g/kg, 1 to 600 mg/kg, 1 to 500 mg/kg, 1 to 400 mg/kg, 1 to 200 mg/kg, 1 to 100 mg/kg, 1 to 90 mg/kg, 1 to 80 mg/kg, 1 to 75 mg/kg, 1 to 70 mg/kg, 1 to 60 mg/kg, 1 to 50 mg/kg, 1 to 40 mg/kg, 1 to 35 mg/kg, 1 to 30 mg/kg, 3 to 30 mg/kg, 3 to 20 mg/kg, 1 to 20 mg/kg, or 1 to 15 mg/kg. In embodiments of the invention, the required dose of a compound disclosed herein administered twice daily is about any of 1 to 50 mg/kg, 1 to 40 mg/kg, 2.5 to 40 mg/kg, 3 to 40 mg/kg, or 3 to 30 mg/kg. In embodiments of the invention, the required daily dose of the compound is about any of 0.01 μg to 5 g/kg, 1 μg to 5 mg/kg, or 1 mg to 1 g/kg and within that range 1 to 500 mg/kg, 1 to 250 mg/kg, 1 to 200 mg/kg, 1 to 150 mg/kg, 1 to 100 mg/kg, 1 to 70 mg/kg, 1 to 65 mg/kg, 2 to 70 mg/kg, 3 to 70 mg/kg, 4 to 65 mg/kg, 5 to 65 mg/kg, or 6 to 60 mg/kg.

In some aspects of the invention, the oral dosage ranges of a scyllo-inositol compound administered once, twice, three times or more daily, in particular once or twice daily, are about any of 1 to 100 mg/kg, 1 to 90 mg/kg, 1 to 80 mg/kg, 1 to 75 mg/kg, 1 to 70 mg/kg, 1 to 60 mg/kg, 1 to 50 mg/kg, 1 to 40 mg/kg, 1 to 35 mg/kg, 2 to 35 mg/kg, 2.5 to 30 mg/kg, 3 to 30 mg/kg, 3 to 20 mg/kg, or 3 to 15 mg/kg.

In embodiments of the invention, the oral dosage ranges for the scyllo-inositol compound are any of about 0.1 mg to about 2 g per kg per day, about 0.5 mg to about 2 g per kg per day, about 1 mg to about 1 g per kg per day, about 1 mg to about 200 mg per kg per day, about 1 mg to about 100 mg per kg per day, about 10 mg to about 100 mg per kg per day, about 30 mg to about 70 mg per kg per day, about 1 mg to about 50 mg per kg per day, about 2 mg to about 50 mg per kg per day, about 2 mg to about 40 mg per kg per day, or about 3 mg to 30 mg per kg per day.

In embodiments of the invention, the required oral dose of scyllo-inositol compound administered twice daily is any of about 1 to about 50 mg/kg, 1 to about 40 mg/kg, 2.5 to about 40 mg/kg, 3 to about 40 mg/kg, 3 to about 35 mg/kg, in particular about 3 to about 30 mg/kg.

In other embodiments of the invention, the required daily dose of scyllo-inositol compound is any of about 1 to about 80 mg/kg and within that range 1 to about 70 mg/kg, 1 to about 65 mg/kg, 2 to about 70 mg/kg, 3 to about 70 mg/kg, 4 to about 65 mg/kg, 5 to about 65 mg/kg, or 6 to about 60 mg/kg.

A medicament or treatment of the invention may comprise a unit dosage of at least one compound of the invention to provide therapeutic effects. A “unit dosage” or “dosage unit” refers to a unitary i.e. a single dose, which is capable of being administered to a patient, and which may be readily handled and packed, remaining as a physically and chemically stable unit dose comprising either the active agents as such or a mixture with one or more solid or liquid pharmaceutical excipients, carriers, or vehicles.

A scyllo-inositol compound can be provided once daily, twice daily, in a single dosage unit or multiple dosage units (i.e., tablets or capsules) having any of about 50 to about 10000 mg, about 50 to about 2000 mg, about 50 to about 1000 mg, about 50 to about 700 mg, about 50 to about 500 mg, about 75 to 600 mg, about 70 to about 7000 mg, about 70 to about 6000 mg, about 70 to about 5500 mg, about 70 to about 5000 mg, about 70 to about 4500 mg, about 70 to about 4000 mg, about 70 to about 3500 mg, about 70 to about 3000 mg, about 75 to 600 mg, about 100 to about 1500 mg, about 150 to about 2500 mg, about 150 to about 2000 mg, about 200 to about 2500 mg, about 200 to about 2000 mg, about 200 to about 1500 mg, about 700 to about 1200 mg, or about 1000 mg, in particular about 200 to 2000 mg, more particularly about 500 to 1200 mg, about 700 to 1000 mg, or about 500 to 1000 mg, most particularly about 500 mg or 1000 mg.

In aspects of the invention, dosages which can be used for systemic administration include, without limitation, an effective amount within the dosage range of about 0.1 μg/kg to about 300 mg/kg, or within about 1.0 μg/kg to about 40 mg/kg body weight, or within about 10 μg/kg to about 20 mg/kg body weight, or within about 0.1 mg/kg to about 20 mg/kg body weight, or within about 1 mg/kg to about 20 mg/kg body weight, or within about 0.1 mg/kg to about 10 mg/kg body weight, or within about 1 mg/kg to about 10 mg/kg body weight, or within about 0.1 μg/kg to about 10 mg/kg body weight.

In aspects of the invention, dosages which can be used for systemic administration when based on body surface area (expressed in square meters, or m²) include, but are not limited to, an effective amount within the dosage range of about 0.1 μg/m² to about 300 mg/m² body surface area, or within about 10 μg/m² to about 300 mg/m² body surface area, or within about 100 μg/m² to about 300 mg/m² body surface area, or within about 1 mg/m² to about 300 mg/m² body surface area, or within about 10 mg/m² to about 300 mg/m² body surface area, or within about 10 mg/m² to about 200 mg/m² body surface area, or within about 10 mg/m² to about 120 mg/m² body surface area, or within about 40 mg/m² to about 120 mg/m² body surface area, or within about 60 mg/m² to about 100 mg/m² body surface area.

In other aspects of the invention for intraocular and intravitreous administration or injection, examples of dosages which can be used include, without limitation, about any of 0.1 μg to 10 mg, 1 μg to 10 mg, 0.1 μg, 1 μg, 5 μg, 10 μg, 15 μg, 20 μg, 25 μg, 30 μg, 50 μg, 75 μg, 100 μg, 200 μg, 300 μg, 400 μg, 500 μg, 600 μg, 700 μg, 800 μg, 900 μg, 1 mg, 2 mg, 3 mg, 4 mg, 5 mg or 10 mg per eye. For periocular administration or injection, examples of dosages which may be used include, without limitation, about any of 0.1 μg to 50 mg, 1 μg to 20 mg, 5 μg to 10 mg, 25 μg to 50 mg, 0.1 μg, 0.5 μg, 1 μg, 5 μg, 10 μg, 15 μg, 25 μg, 50 μg, 100 μg, 150 μg, 200 μg, 250 μg, 300 μg, 350 μg, 400 μg, 500 μg, 600 μg, 700 μg, 750 μg, 800 μg, 900 μg, 1 mg, 1.5 mg, 2 mg, 2.5 mg, 3 mg, 3.5 mg, 4 mg, 4.5 mg, 5 mg, 6 mg, 7 mg, 8 mg, 9 mg, 10 mg, 12.5 mg, 15 mg, 20 mg, 25 mg, 30 mg, 40 mg, or 50 mg per eye.

A subject may be treated with a scyllo-inositol compound or medicament thereof on substantially any desired schedule. A scyllo-inositol compound or medicament of the invention may be administered one or more times per day, in particular 1 or 2 times per day, once per week, once a month or continuously. However, a subject may be treated less frequently, such as every other day or once a week, monthly or more frequently. A scyllo-inositol compound or medicament may be administered to a subject for about or at least about 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, 2 weeks to 24 months, or for more than 24 months, periodically or continuously.

In embodiments, the methods for the treatment of age-related macular degeneration disorders comprise administering a scyllo-inositol compound about every week, about every other week, or about every 4, 5, 6, 7, 8, to 10 or 11 to 14 days to a subject in need thereof. In one embodiment, a scyllo-inositol compound can be administered about every 7 days. In particular embodiments, administration can be a one-time administration every three or six months or every year. In particular embodiments, the administration can continue for about 1 week, about 2 weeks, about 3 weeks, about 4 weeks, about 5 weeks, about 6 weeks, about 7-12 weeks, about 12-24 weeks, or about 24 to 52 weeks. In certain embodiments, administration of the scyllo-inositol is about every 7 days for about 5 weeks. In particular embodiments, administration may be intermittent. For example, a patient may be treated once a week for about 4-8 weeks and then treated about 3-6 times over the following year.

In an embodiment, dosages of scyllo-inositol compounds may be administered in a sustained release formulation or a sustained release implant including an implant which gradually releases the compounds over a period of time and which allows the compounds to be administered less frequently, for example once a month, about once every 2-6 months, about once every year, or even a single administration which need not be repeated. Sustained release implants, devices or formulations may be administered by topical application to the eye by injection, or can be surgically implanted in various locations in the eye or tissues associated with the eye, such as intraocular, intravitreal, vitreous chamber, vitreous body, subretinal, periocular, retrobulbar, subconjunctival or subtenons. A sustained release formulation may be combined with iontophoretic methods. Sustained release formulations including formulations suitable for ocular delivery which may be used in various embodiments of the present invention are described, for example, in U.S. Pat. Nos. 6,713,081, 6,692,759, 6,331,313, 5,869,079, 5,824,072, US Published Application Nos. 20050048099, 20050281861, 20080089923, and 20070160592, and published PCT Application No. WO2007065149.

For combination treatments or compositions, the dosages used for the second agent or therapeutic may be similar to those dosages known to those skilled in the art and used in pre-clinical and clinical studies and in commercial use. The concentrations may be lower than the currently used dosages as the combination of the agents may increase efficacy of one or more of the agents. For example, a scyllo-inositol may be combined with an anti-vascular endothelial growth factor (anti-VEGF) therapeutic or an anti-oxidant, with the objective to reduce the dosages and/or reduce frequency of administration of the anti-vascular endothelial growth factor (anti-VEGF) therapeutic or anti-oxidant, in order to achieve both effective treatment and to lessen any negative effects of the anti-vascular endothelial growth factor (anti-VEGF) therapeutic or anti-oxidant.

A particular dosage of a scyllo-inositol compound for combination treatments of the invention may be the maximum a patient requires to provide an optimal enhancing effect (e.g. synergistic effect), such maximum being tempered by the absolute upper limit of scyllo-inositol compound dosage being the maximum that a subject can tolerate and not develop any serious complications. Those skilled in the art will be aware that the amounts of the various components of the compositions and combination treatments of the invention to be administered in accordance with the invention to a patient will depend upon those factors noted above.

The following are examples of regimens for combination treatments with conventional treatments:
1. More than once daily, daily, more than once weekly, weekly, more than once monthly or monthly administration of a scyllo-inositol compound in combination with an anti-vascular endothelial growth factor (anti-VEGF) therapeutic, an anti-oxidant, photocoagulation therapy or photodynamic therapy for the effective treatment of a macular degeneration related disorder;
2. More than once daily, daily, more than once weekly, weekly, more than once monthly or monthly administration of a scyllo-inositol compound simultaneously with an anti-vascular endothelial growth factor (anti-VEGF) therapeutic, an anti-oxidant, photocoagulation therapy or photodynamic therapy for the effective treatment of a macular degeneration related disorder;
3. More than once daily, daily, more than once weekly, weekly, more than once monthly or monthly treatments with a scyllo-inositol compound and an anti-vascular endothelial growth factor (anti-VEGF) therapeutic, an anti-oxidant, photocoagulation therapy or photodynamic therapy administered separately either more than once daily, daily, more than once weekly, weekly, more than once monthly, or monthly;
4. More than once daily, daily, more than once weekly, weekly, more than once monthly or monthly administration of a scyllo-inositol compound and an anti-vascular endothelial growth factor (anti-VEGF) therapeutic, an anti-oxidant, photocoagulation therapy or photodynamic therapy as well as adjunct administration of more than once daily, daily, more than once weekly, weekly, more than once monthly or monthly doses of a scyllo-inositol compound; and
5. More than once daily, daily, more than once weekly, weekly, more than once monthly or monthly treatments with a scyllo-inositol compound, a combination of a scyllo-inositol compounds and an anti-vascular endothelial growth factor (anti-VEGF) therapeutic, an anti-oxidant, photocoagulation therapy or photodynamic therapy, and a combination of a scyllo-inositol compound and an anti-vascular endothelial growth factor (anti-VEGF) therapeutic, an anti-oxidant, photocoagulation therapy or photodynamic therapy administered separately either more than once daily, daily, more than once weekly, weekly, more than once monthly, or monthly.

### Diagnostics

In embodiments, the invention provides methods for diagnosing a macular degeneration-related disorder in a subject by detecting β-amyloid in ocular tissue from the subject with a scyllo-inositol compound. In some embodiments, the present invention provides a method of diagnosing a macular-degeneration-related disorder in a subject comprising: (a) collecting a sample from the subject; (b) contacting the sample with a scyllo-inositol compound tagged or labelled with a detectable substance that emits a detectable signal when the scyllo-inositol compound binds to β-amyloid if present; and (c) detecting the detectable signal from the scyllo-inositol compound bound to the β-amyloid in the sample to determine the level of β-amyloid in the sample; and, (d) comparing to a standard wherein altered levels of β-amyloid relative to the corresponding standard is an indication that the patient is afflicted with an age-related macular degeneration disorder. A standard may correspond to levels quantitated for samples from control subjects with no disease or early stage disease or from other samples of the subject.

In embodiments, the invention provides methods for assessing a response to therapy in a subject suffering from a macular degeneration-related disorder by detecting β-amyloid in ocular tissue from the subject with a scyllo-inositol compound. In embodiments, the present invention provides a method for assessing efficacy of a treatment in a subject suffering from a macular degeneration-related disorder comprising: (a) collecting samples from the subject before and after the treatment; (b) contacting the samples with a scyllo-inositol compound tagged or labeled with a detectable substance that emits a detectable signal when the scyllo-inositol binds to β-amyloid if present; and (c) detecting the detectable signals from the scyllo-inositol bound to the β-amyloid in the samples to determine the levels of β-amyloid in the samples; and, d) comparing the levels of β-amyloid in the samples collected before or after treatment to a standard, wherein a difference in the levels of β-amyloid in the sample before and after treatment is indicative of the efficacy of the treatment. In certain embodiments, the levels of β-amyloid in the samples following treatment are lower or reduced compared to the levels in samples before treatment and are indicative that the treatment is efficacious.

A standard may correspond to levels quantitated for samples from control subjects with no disease or early stage disease or from other samples of the subject.

Examples of detectable substances include, but are not limited to, the following: radioisotopes (e.g., ³H, ¹⁴C, ³⁵S, ¹²⁵I, ¹³¹I); fluorescent labels, (e.g., FITC, rhodamine, lanthanide phosphors); luminescent labels such as luminol; enzymatic labels (e.g., horseradish peroxidase, beta-galactosidase, luciferase, alkaline phosphatase, acetylcholinesterase); biotinyl groups (which can be detected by marked avidin e.g., streptavidin containing a fluorescent marker or enzymatic activity that can be detected by optical or calorimetric methods); and predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. In embodiments, the detectable signal is a fluorescent or an enzyme-linked immunosorbent assay signal.

In embodiments of the diagnostic methods of the invention, the scyllo-inositol compound is 2-[¹⁸F]fluoro-2-deoxy-scyllo-inositol.

In embodiments of the diagnostic methods of the invention, the scyllo-inositol compound is [¹⁸F]-1-deoxy-1-fluoro-scyllo-inositol.

The diagnostic methods encompass detection of abnormality in β-amyloid in ocular tissue. Typically a diagnostic method works by comparing a measured level of β-amyloid in a subject with a baseline level determined in a control population of subjects unaffected by a macular degeneration-related disorder. The outcome of the diagnostic test may be considered negative if the measured level is not significantly different from the baseline level in a control population. The outcome of the diagnostic test may be considered positive if there is no significant departure between the measured level in a subject and the baseline level in unaffected subjects. A departure may be considered significant if the measured level falls outside the range typically observed in unaffected subjects due to inherent variation between subjects and experimental error. In some embodiments of the diagnostic methods, a significant departure occurs when the measured level does not fall within the mean plus one standard deviation of the baseline levels in unaffected subjects. In some embodiments, a significant departure occurs when the difference between the measured level and baseline level is at least about 10%, 20%, 30%, 40%, 50%, 60%, 70%, 80% or 90%.

A subject that has a positive outcome in a diagnostic method disclosed herein may be at a minimum susceptible to, or at risk of a macular degeneration-related disorder and accordingly may be subjected to further tests or screening to confirm the initial diagnosis. For example, the subject may be screened for C5b-9 complex in choriocapillaris, complement pathway molecules, genetic markers (e.g. 1p21-q13 for recessive Stargardt's disease or fundus flavi maculates, 1q25-q31 for recessive AMD, 6p21.2-cen for dominant macular degeneration, and adult vitelloform), and drusen-associated phenotypic markers (e.g. RPE dysfunction and/or death, immune mediated events, dendritic cell activation, migration and differentiation, extrusion of the dendritic process into the sub RPE space, and the presence of geographic atrophy or disciform scars) or drusen-associated genotypic markers that correlate with macular degeneration-related disorders (e.g. CD68, CD1a, HLA-DR, apolipoprotein E, clusterin and S100). Additional screening can include monitoring clinical symptoms including the presence of drusen and retinal pigmentary changes.

The invention will be described in greater detail by way of a specific example. The following example is offered for illustrative purposes, and is not intended to limit the invention in any manner.

### Example

The ocular pathologies of AMD may be recapitulated in a murine model by applying three physiologically relevant risk factors: specific APOE genotype (APOE4), advanced age and high fat/cholesterol-rich (HF-C) diet. These mice develop sub-retinal pigment epithelium (RPE) deposits (basal deposits), RPE atrophy and choroidal neovascularization in a temporal, non-fully penetrant manner that is analogous to human AMD progression [Malek, G., et al., (2005), Proc Natl Acad Sci USA 102, 11900-5]. An electrophysiological phenotype is associated with this pathology. Electroretinogram (ERG) recordings of APOE4 HF-C mice demonstrate statistically significant decreased a- and b-wave amplitudes [Ding J D et al, (2008), Vision Res. 48(3):339-45]. The ability of scyllo-inositol (AZD-103/ELN005) to prevent retina/RPE damage, the buildup of basal deposits and attenuation of the ERG was evaluated.

Aged male APOE4 mice housed conventionally, under ambient conditions maintained on water ad libitum and normal mouse chow (normal diet or ND), were assigned to three treatment groups. This assignment was random, although the ages of the animals were balanced across the groups. One group was maintained on the normal diet. The second group was switched to a high fat cholesterol (HFC) diet (35% fat, 20% protein, 45% carbohydrates, 1.25% cholesterol, 0.5% sodium cholate) for 8 weeks. The third group received scyllo-inositol (AZD-103/ELN005) ad libitum (dissolved in drinking water at 10 mg/ml) for 7 days. They were then switched to the HF-C diet for 8 weeks, during which time they continued to receive scyllo-inositol (AZD-103/ELN005) ad libitum. Animals underwent assessments prior to dietary assignment, and after 8 weeks on the assigned diet. After this time all animals were sacrificed. The animals underwent the following assessments:
1. Fundus examination and photography before and after the assigned diet.
2. Total plasma cholesterol levels in whole blood of fasted animals before and after the assigned diet.
3. Full-field ERGs before and after the assigned diet. Animals were dark adapted for at least 12 hours. Each animal was anesthetized with a ketamine/xylazine cocktail, pupils dilated and the animal stabilized on a 37° C. warming pad. ERG tracings were recorded using a platinum iridium wire loop electrode placed in contact with the eye along with a drop of 2.5% hydroxypropyl methylcellulose. Mice were placed in a photopic stimulator chamber where the animal was exposed to flashes of light (max intensity of 1000 cd-s/m² attenuate in 1 log steps, starting from 0.0005). The a-wave amplitude was measured from baseline to the a-wave trough, and the b-wave amplitude was measured from the a-wave trough to the b-wave peak.
4. Postmortem immunohistochemical localization of proteins, including amyloid beta, other proteins associated with AMD lesions (vitronectin, apoE, apoB), and proteins associated with photoreceptor synaptic terminals: SV2 VGLUT1, PKCα.
5. Quantitation of photoreceptors. Eyes were fixed and embedded in Epon-Spurr resin, cut at 500 nm and mounted on glass slides. Cross sections that bisect the optic nerve were used for measurement of retinal layers and to count cell numbers. The thickness of outer nuclear layer and the linear density of photoreceptor will be calculated.

### Results:

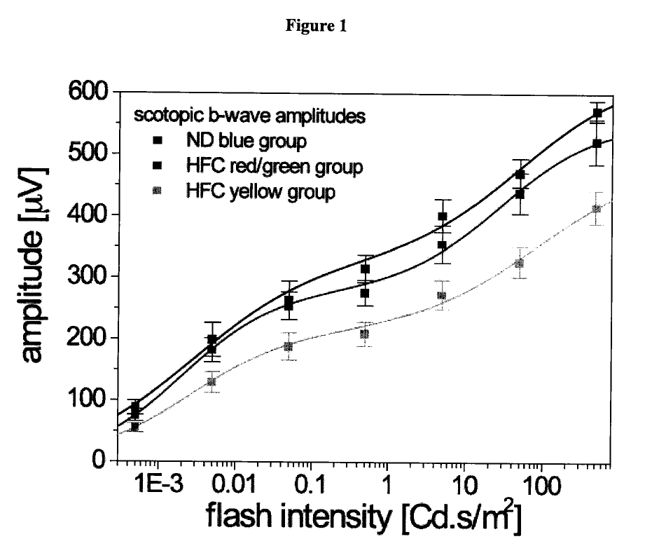

ApoE4 mice were aged to 12 months. At this point animals were assigned to one of three groups:
Normal diet (black line in FIG. 1)
High cholesterol diet plus regular drinking water (green line in FIG. 1)
High cholesterol diet plus scyllo-inositol (AZD-103/ELN005) in drinking water (red line in FIG. 1)

After 8 weeks, animals were assessed by electroretinogram. Each animal was exposed to flashes of increasing intensity. The electrophysiological response to these flashes was recorded by an electrode touching the surface of the eye. A- and b-waves were plotted. The b-wave showed the greatest amplitude, so data on this endpoint are presented in FIG. 1 (results of assessment of the a-wave are consistent). Animals which were switched to the high cholesterol diet, and which received vehicle treatment, showed a reduction in the b-wave amplitude at all flash intensities. In contrast, animals receiving a high cholesterol diet together with scyllo-inositol (AZD-103/ELN005) in drinking water showed b-wave amplitudes which were virtually indistinguishable from the normal diet control animals. Scyllo-inositol (AZD-103/ELN005) treatment therefore prevented the retinal defect that was caused by high cholesterol diet. In conclusion, AMD animals treated with scyllo-inositol (AZD-103/ELN005) had preserved retinal function as demonstrated by ERGs.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the methods etc. which are reported therein which might be used in connection with the invention. Nothing herein is to be construed as an admission that the invention is not entitled to antedate such disclosure by virtue of prior invention.

What is claimed is:

1. A medicament for use in treating a macular degeneration-related disorder comprising a therapeutically effective amount of a scyllo-inositol compound of the Formula Ia, Ib: 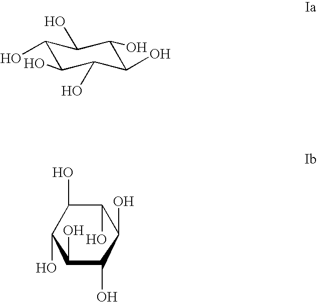
or Formula Ia or Ib wherein one or more of the hydroxyl groups are replaced by substituents with retention of configuration wherein the substituents are selected from the group consisting of hydrogen; alkyl; substituted alkyl; acyl; alkenyl; substituted alkenyl; alkynyl; substituted alkynyl; alkoxy; substituted alkoxy; halogen; —NHR¹ wherein R¹ is hydrogen, acyl, alkyl or —R²R³ wherein R² and R³ are the same or different and represent acyl or alkyl; —PO₃H₂; —SR⁴ wherein R⁴ is hydrogen, alkyl, or —O₃H; and —OSO₃H.

2. A medicament according to claim 1 wherein in the Formula Ia or Ib one or two of the hydroxyl groups is replaced with C1-C4 alkyl, chloro or fluoro; alkenyl; C1-C4 alkoxy; or, —NHR¹ wherein R¹ is hydrogen or alkyl.

3. A medicament according to claim 1 wherein in the Formula Ia or Ib one of the hydroxyl groups is replaced with C1-C4 alkyl.

4. A medicament according to claim 1 wherein in the Formula Ia or Ib one of the hydroxyl groups is replaced with C1-C4 alkoxy.

5. A medicament according to claim 1 wherein in the Formula Ia or Ib one of the hydroxyl groups is replaced with halogen.

6. A medicament according to claim 1 having the structure of Formula Ia or Ib.

7. A medicament according to any one of claims 1 to 6 which is in a form suitable for administration to the surface or interior of the eye.

8. A medicament according to claim 7 which is in the form of an ophthalmic depot formulation for subconjunctival administration.

9. A medicament according to claim 7 comprising a pharmaceutically acceptable carrier, excipient or vehicle for intravitreal administration.

10. A method for improving ocular function and/or slowing degeneration of ocular tissue in a subject after the onset of symptoms of a macular degeneration-related disorder comprising administering an effective amount of a scyllo-inositol compound according to claim 1 and a pharmaceutically acceptable carrier, excipient, or vehicle.

11. A method for treating a macular degeneration-related disorder in a subject comprising administering an effective amount of a scyllo-inositol compound as defined in claim 1 and a pharmaceutically acceptable carrier, excipient, or vehicle.

12. The method of claim 11 wherein the a scyllo-inositol compound as defined in claim 1 has the structure of Formula Ia or Ib.

13. A method for delaying one or more disability of a macular degeneration-related disorder in a subject comprising administering a therapeutically effective amount of a scyllo-inositol compound as defined in claim 1 and a pharmaceutically acceptable carrier, excipient, or vehicle.

14. A method for preventing and/or ameliorating symptoms or periodicity of recurrence of a macular degeneration-related disorder in a subject comprising administering to the subject a therapeutically effective amount of a scyllo-inositol compound as defined in claim 1 and a pharmaceutically acceptable carrier, excipient, or vehicle.

15. A method according to any one of claim 10, 11, 12, 13 or 14 wherein the subject is a human subject with age-related macular degeneration in one or both eyes.

16. A method according to claim 15 wherein the age-related macular degeneration is wet macular degeneration.

17. A method according to claim 15 wherein the age-related macular degeneration is choroidal neovascularization secondary to age-related macular degeneration.

18. A method according to claim 12 wherein the subject has neovascular age-related macular degeneration characterized by classic, occult and mixed lesions of up to 12 disc areas and baseline visual acuity in an eye between 20/40 and 20/320.

19. A method according claim 12 wherein the subject has one or more of the following: visual acuity loss of greater than 5 letters, evidence of fluid in the macula by optical coherence tomography (OCT), an increase in OCT central retinal thickness of greater than 100 mM, existing or new macular hemorrhage, an existing or new area of classic choroidal neovascularization, and persistent fluid by OCT.

20. A method according to claim 12 wherein the subject is or has been treated with an anti-VEGF therapeutic.

21. A method according to claim 15 wherein the subject suffered a relapse following cessation of administration of the anti-VEGF therapeutic or suffered a relapse while on the anti-VEGF therapeutic.

22. A method according to claim 21 wherein the anti-VEGF therapeutic is pegaptanib sodium or ranibizubmab.

23. A method of treating a macular degeneration-related disorder in a subject that has had a sub-optimal response to treatment with an agent chosen from one or more of an anti-vascular endothelial growth factor (anti-VEGF) therapeutic, an anti-oxidant, photocoagulation therapy and photodynamic therapy, comprising administering to the subject an effective amount of a scyllo-inositol compound as defined in claim 1 and optionally, an anti-vascular endothelial growth factor (anti-VEGF) therapeutic, an anti-oxidant, photocoagulation therapy or photodynamic therapy to provide an enhanced or optimal response.

24. A method of retreatment of a subject suffering from a macular degeneration-related disorder that has one or more adverse events with an anti-vascular endothelial growth factor (anti-VEGF) therapeutic, an anti-oxidant, photocoagulation therapy or photodynamic therapy comprising administering to the subject a therapeutically effective amount of a scyllo-inositol as defined in claim 1.

25. A kit comprising at least one medicament according to claim 1, a container, and instructions for treating a macular degeneration-related disorder.
